(19) United States
(12) Patent Application Publication
(10) Pub. No.: US 2003/0176697 A1
(43) Pub. Date: Sep. 18, 2003

(54) Method for preparing crambescidin core acid intermediates and their use for preparing crambescidin alkaloid analogs as therapeutic agents

(75) Inventors: Larry E. Overman, Corona del Mar, CA (US); Frank Stappenbeck, San Francisco, CA (US); Andrew I McDonald, San Francisco, CA (US); Zachary D. Aron, Irvine, CA (US)

Correspondence Address: Robert R. Buyan, Stout, Uxa, Buyan & Mullins, LLP, Suite 300, 4 Venture, Irvine, CA, 92618, US

(73) Assignee: The Regents of the University of California

(21) Appl. No.: 10/255,994

(22) Filed: Sep. 24, 2002

Related U.S. Application Data
(63) Continuation-in-part of application No. 10/018,630, filed on Dec. 14, 2001.
Application No. 10/018,630 is a 371 of international application No. PCT/US00/18395, filed on Jun. 30, 2000.
(60) Provisional application No. 60/142,027, filed on Jun. 30, 1999.
(60) Provisional application No. 60/142,028, filed on Jun. 30, 1999.

(51) Int. Cl. A61K 31/519; C07D 487/14

(52) U.S. Cl. 544/231; 514/257

(57) ABSTRACT

The invention provides methods to synthesize zwitterionic pentacyclic crambescidin core intermediates having the carboxylate side chain in the natural axial orientation, and a range of crambescidin alkaloid analogs.

This application is a continuation-in-part of U.S. Ser. No. 10/018,630, which is based on International Application PCT/US00/18395, filed Jun. 30, 2001, which claims priority to Provisional applications 60/142,027, and 60/142,028, filed Jun. 30, 1999, the contents of which are incorporated by reference in their entirety into the present application.

Throughout this application various publications are referenced. The disclosures of these publications in their entireties are hereby incorporated by reference into this application in order to more fully describe the state of the art to which this invention pertains.

### FIELD OF THE INVENTION

The present invention relates to methods and compositions for preparing pentacyclic intermediates and the methods of using them to prepare therapeutic natural and non-natural side chain analogs of the crambescidin/ptilomycalin family of guanidinium alkaloids.

### BACKGROUND OF THE INVENTION

Crambe crambe, a bright red encrusting sponge commonly found at shallow depths along the rocky coast of the Mediterranean is a rich source of structurally novel, bioactive alkaloids (FIG. 1).

Among the most remarkable marine guanidine natural products are the family of alkaloids depicted in FIG. 1 that have a rigid pentacyclic guanidine carboxylic acid core linked to an ω-hydroxycarboxylic acid, ester or polyamine amide. This family, exemplified by Ptilomycalin A (compound 1), the Crambescidins (compounds 2-4, and 6-8), Celeromycalin (compound 5) and Neofolispates (compound 7) are characterized by a structurally unique pentacyclic guanidinium core that has a spermidine or hydroxyspermidine residue tethered by a long chain ω-hydroxycarboxylic acid spacer.

The alkaloid, Ptilomycalin A, was reported by Kashman, Kakisawa and co-workers from sponges collected in the Caribbean and Red Sea (Kashman et al., J. Am. Chem. Soc., 1989, 111, 8925). Ptilomycalin A exhibits cytotoxicity against P388 (IC₅₀ 0.1 μg/mL), L1210 (IC₅₀ 0.4 μg/mL) and KB (IC₅₀ 1.3 μg/mL), antifungal activity against Candida albicans (MIC 0.8 μg/mL) as well as considerable antiviral activity against Herpes simplex virus, type 1 (HSV-1) at a concentration of 0.2 μg/mL (Overman, L. E.; et al. infra). Recently, Ptilomycalin A has been shown to inhibit the brain Na⁺, K⁺-ATPase and Ca²⁺-ATPase from skeletal sarcoplasmic reticulum with IC₅₀ values of 2 μM and 10 μM, respectively (Ohtani, I.; et al. Euro. J. Pharm. 1996, 310, 95).

In addition to Ptilomycalin A, numerous other complex marine alkaloids having a hydropyrrolo[1,2-c]pyrimidine-4-carboxylate part structure have been isolated including 13,14,15-Isocrambescidin 800, Crambescidin 800 and Crambescidin 816 from Crambe crambe (Jares-Erijman et al., J. Org. Chem. 1991, 56, 5712-5715; Jares-Erigman et al., J. Org. Chem. 1993, 58, 4805-4808; Tavares et al., Biochem. Syst. Ecol., 1994, 22, 645-646; Berlinck et al., J. Nat. Prod. 1993, 56, 1007-10015.)

Ptilomycalin A and several of the Crambescidins show substantial antitumor, antiviral and antifungal activities. Crambescidin alkaloids have been described for use in inhibition of calcium channels (Jares-Erijman, et al., J. Org. Chem. 1993, supra); inhibition of Na⁺, K⁺ and Ca²⁺-ATPases (Ohizumi et al., Eur. J. Pharmacol., 1996, 310, 95). Batzelladine alkaloids, exemplified by Batzelladines B and D (FIG. 1, Patil et al., J. Org. Chem., 1995, 60, 1182; Patil et al., J. Org. Chem., 1997, 62, 1814; and Patil et al., J. Nat. Prod., 1997, 60, 704), are reported to modulate protein-protein interactions that are important for immunological responses (Patil et al., 1995 and J. Org. Chem., 1997, supra). Comparison of the cytotoxicity profile of ptilomycalin A with compounds in the NCI database suggests that these alkaloids may have a unique mode of action (Boyd, M. R.; Paull, K. D. Drug. Dev. Res. 1995, 34, 91-109).

As a result of its low abundance, 13,14,15-Isocrambescidin 800 has not been extensively screened, although it is reported to be less cytotoxic to L-1210 cells than other Crambescidins. (Jares-Erijman et al., J. Org. Chem., 1993, supra).

The defining structural feature of the crambescidin alkaloids is a pentacyclic guanidine unit linked by a straight chain ω-hydroxycarboxylic acid tether to a spermidine or hydroxyspermidine unit. Extensive NMR studies demonstrated that the relative stereochemistry of the pentacyclic cores of Crambescidin 800, Crambescidin 816 and Ptilomycalin A is identical (Jares-Erijman et al., supra and Tavares et al., supra), while 13,14,15-Isocrambescidin 800 is epimeric at C13, C14 and C15 relative to other members of the Crambescidin family (Jares-Erijman et al., J. Org. Chem., 1993, supra, and Berlinck et al., J. Nat. Prod., supra). The absolute configuration of the guanidine moieties of 13,14,15-Isocramescidin 800 and Crambescidin 816 was established by oxidative degradation of the oxepene rings of these alkaloids to yield (S)-2-hydroxybutanoic acid (Jares-Erijman et al., J. Org. Chem., 1993, supra), while the absolute configuration of the hydroxyspermidine unit of crambescidin 816 was assigned using Mosher's method (Berlinck et al., supra, and Dale et al., J. Am. Chem. Soc., 1973, 95, 512-519). Since ¹H NMR and ¹³C NMR chemical shifts in the hydroxyspermidine fragments of 13,14,15-Isocrambescidin 800 are nearly identical to those of 2 and 3, it had been assumed that the stereochemistry at C14 is the same for all Crambescidins (Berlinck et al., supra).

Apparent in the alkaloid compounds described (FIG. 1), is the occurrence of the hydropyrrolo[1,2c]pyrimidine unit with either the syn or anti relationship of the hydrogens flanking the pyrrolidine nitrogen.

In 1893, Biginelli reported the synthesis of dihydropyrimidines from the condensation of ethyl acetoacetate, aromatic aldehydes and urea. (Biginelli, P., Gazz. Chem. Ital., 1893, 23, 360. Since Biginelli's disclosure, variations in all three components have led to the synthesis of an array of functionalized dihydropyrimidines and analogues. (Kappe, C. O., Tetrahedron, 1993, 49, 6937.

In 1993, we reported on the viability of “tethered Biginelli” condensations and verified that the cis orientation of the methine hydrogens was preferentially realized when the dehydrative condensation was promoted under Knoevenagel conditions to form Cis-1-oxohexahydropyrollo[1,2-c]pyrimidine products. (Overman et al., J. Org. Chem., 1993, 58, 3235-3237). These reactions represented the first use of the Biginelli reactions in stereocontrolled organic synthesis. Tethered Biginelli condensations have already proved to be powerful reactions for the construction of Crambescidin (Overman et al., J. Am. Chem. Soc., 1995, 117, 265) and Batzelladine alkaloids (Franklin et al., J. Org. Chem., 1999, 62, 6379). Recently it was reported to use acetals in place of alkenes to generate the aldehyde component of a Biginelli cyclization (Cohen et al., Organic Letters, 1999, 1 2169-2172).

In 1995, an enantioselective total synthesis of (−)-Ptilomycalin A (Overman et al., J. Am. Chem. Soc., 1995, 117, 2657) was reported, which was the first total synthesis of a member of the Crambescidin alkaloid family.

A method for chemical synthesis of the Crambescidin/Ptilomycalin family of alkaloids has been described (U.S. Ser. No. 10/018,630 and PCT/US00/18395), where this chemistry was used to prepare allyl ester of the pentacyclic alkaloid and its deprotection to form core acid, in which the ester and acid substituents at C14 was assumed to be in the unnatural equatorial orientation.

There remains a need for improved methods to produce pentacyclic core intermediates that can in turn be utilized for divergent synthesis of natural and non-natural side chain Crambescidin/Ptilomycalin alkaloid analogs having therapeutic activity, such as antifungal, antiviral and/or anti-tumor activity.

### SUMMARY OF INVENTION

The present invention provides compositions and improved methods for the total synthesis of the Crambescidin/Ptilomycalin family of novel alkaloid analogs of Formula 11, for use as therapeutic agents having antitumor, antiviral and/or antifungal activity. The novel compounds of the invention have the carboxylate side chain at C14 in the natural axial position.

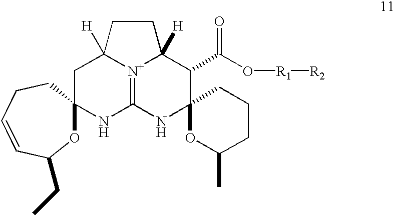

where R₁ is selected from a saturated or unsaturated, cyclic or acyclic, straight or branched, chiral or achiral hydrocarbyl group with from 1 to 20 carbon atoms and optionally one or more carbons in the said hydrocarbyl group may be replaced with one or more elements selected from O, S and NR₃, where R₃ is alkyl, cycloalkyl or acyl; additionally any one or more carbons in R₁ may be substituted with alkyl, aryl, aralkyl, heteroalkyl and heteroaralkyl group, which may be further substituted with one or more groups selected from halo, nitro, cyano, trifluoromethyl, hydroxy, thio, methylthio, amino, substituted amino, acylamino, aminoalkylamino, guanidino, carboxyl, carboalkoxy and the like; furthermore any one carbon in R₁ may be optionally substituted with an aryl or heteroaryl group with from 6 to 14 carbon atoms, including but not limited to phenyl, naphthyl, biphenylyl, furyl, thienyl, pyrrolyl, imidazolyl, benzofuranyl, benzoxazolyl, benzothiazolyl, thianaphthyl, and indolyl.

R₂ is H, alkyl, aryl, heteroaryl, optionally substituted with one or more groups selected from halo, nitro, cyano, trifluoromethyl, hydroxy, thio, methylthio, amino, substituted amino, acylamino and guanidino, or is selected from a group consisting of carboxy, carboxylate anion, phosphonate, phosphate, sulphonate, sulphate, borate, boronate, amine such as NR₄R₅, or carboxamide —CO—NR₄R₅, where R₄ and R₅ are selected from H, alkyl, aralkyl, carboxyalkyl, amino-iminomethyl of the formula —C═N—R₆(—NR₇);carboxyalkyl substituted at the alkyl carbon by hydroxyalkyl, alkyl, thioalkyl, aminoalkyl, carboxyalkyl, amidoalkyl and guanidinoalkyl; hydroxyalkyl, acyl, aminoalkyl, optionally substituted with one or more hydroxy groups, aminoalkylaminoalkyl, hydroxyalkylaminoalkyl and acyl derivatives thereof or R₄ and R₅ may combine to form a cyclic ring, optionally substituted with another heteroatom such as O (morpholino) or NR₈, where R₈ is H (piperazino) or alkyl (alkyl piperazino), carboxyalkyl, hydroxyalkyl or aminoalkyl; R₆ and R₇ are selected from H and alkyl or they may be bridged with 2 to 4 carbons to form a cyclic structure; or R₁ and R₂ and X are absent to offer the pentacyclic C14 carboxylate zwitterion.

The compounds of Formula 11 further include pharmaceutically acceptable salts with organic and inorganic acids, organic and inorganic bases or may be in the form of a zwitterion.

In one embodiment, the invention provides the synthesis, isolation, purification and full characterization of a pentacyclic zwitterionic core acid (compound 10), having the carboxylate side chain at C14 in the natural axial position.

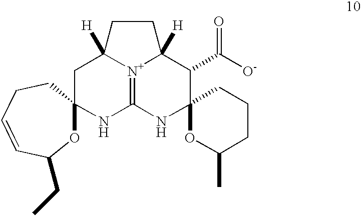

The invention further provides methods of coupling the zwitterionic core acid (compound 10) with side chain —R₁—R₂ by reacting the zwitterionic acid with an alcohol (HO—R₁—R₂) or an iodide (I—R₁—R₂) to provide pentacyclic guanidinium alkaloid analogs covered by Formula 11.

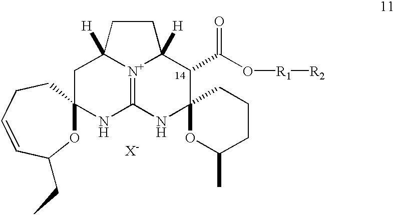

where, for example, R₁=alkyl, such as ethyl; alkenyl, such as propenyl (allyl), and R₂ is aryl, such as phenyl; or

R₁ is —CH₂—(CH₂)n, wherein n=1-20;

and R₂ is —CO—O^{—} (carboxylate group), or

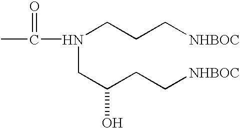

wherein BOC is tert. Butoxycarbonyl, an amine protecting group.

Accordingly, the invention provides compositions and methods of making Crambescidin 431 (compound 19) and analogs;

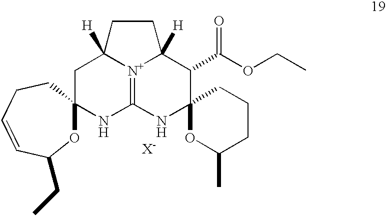

and related ester analog Compound 18

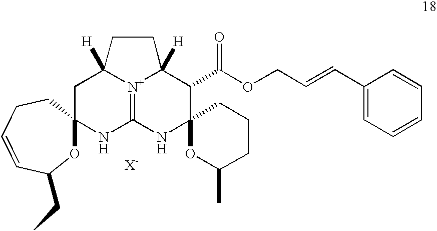

where, X=any pharmaceutically acceptable counterion.

The invention also provides compositions and methods of making Crambescidin analogs of Formula 11, where R₁ is —(CH₂—CH₂—O—)ₙ and R₂ is alkyl.

Specifically, the invention provides composition and methods of synthesizing compound, where R₁ is —(CH₂—CH₂—O—)ₙ, R₂ is ethyl and n is 1-6.

The invention also provides compositions and methods of making Crambescidin analogs of Formula 11, where R₁ is —CH₂—(CH₂)ₙ— and R₂ is a polar group consisting of carboxy, carboxylate anion, phosphonate, phosphate, sulphonate, sulphate, borate, boronate and an amine such as NR₄R₅, or carboxamide —CO—NR₄R₅, where R₄ and R₅ are selected from H, alkyl, aralkyl, carboxyalkyl, amino-iminomethyl of the formula —C═N—R₆(—NR₇), where R₆ and R₇ are selected from H and alkyl, or they may be bridged with 2 to 4 carbons to form a cyclic structure.

Specifically, n is selected from 1-20 and R₂ is carboxy, carboxylate anion, or carboxamide —CO—NR₄R₅, where R₄ and R₅ are selected from H and alkyl, or amino-iminomethyl of the formula —C═N—R₆(—NR₇), where R₆ and R₇ are selected from H and alkyl or they may be bridged with 2 carbons to form a cyclic structure.

More specifically, the invention provides compositions and methods of making Crambescidin 657 analogs of the following formula:

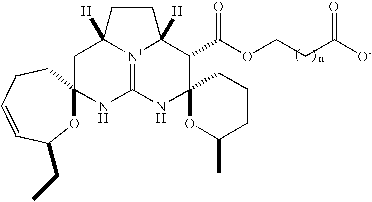

where, n=1-20.

The invention further provides compositions and methods of making Crambescidin 800 analogs described in Formula 11, where R₁ is —CH₂—(CH₂)ₙ— and R₂ is a polar group such as carboxamide —CO—NR₄R₅, where R₄ and R₅ are selected from aminoalkyl, optionally substituted with one or more hydroxy groups as exemplified by the structure below.

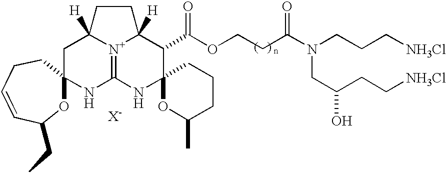

where, n=1-20; X=any pharmaceutically acceptable counterion.

The methods of the invention employ methods for obtaining the compounds of the invention and their use as therapeutic agents.

### BRIEF DESCRIPTION OF THE FIGURES

FIG. 1 depicts structures of naturally occurring pentacyclic guanidinium Ptilomycalin/Crambescidin alkaloids, Ptilomycalin A (compound 1, R¹═R²═R³═H; n=10); Crambescidin 800 (compound 2, R¹═R²═H; R³=α-OH; n=10); Crambescidin 816 (compound 3, R¹═OH, R²═R³=α-OH; n=10); Crambescidin 844 (compound 4, R¹═R³═OH, R²═H; n=12); Celeromycalin (compound 5, R¹═R³═H, R²=β-OH; n=10); Crambescidin 657 (compound 7, R═O^{—}); Neofolispates (compound 7, R═OMe), and Crambescidin 830 (compound 8, R¹═R³═OH, R²═H; n=11).

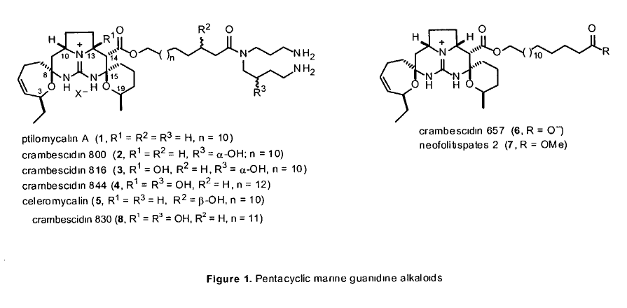

FIG. 2 depicts the synthesis of zwitterionic crambescidin core acid (compound 10) having the carboxylate side chain in the natural axial position, as described in Example 1, infra.

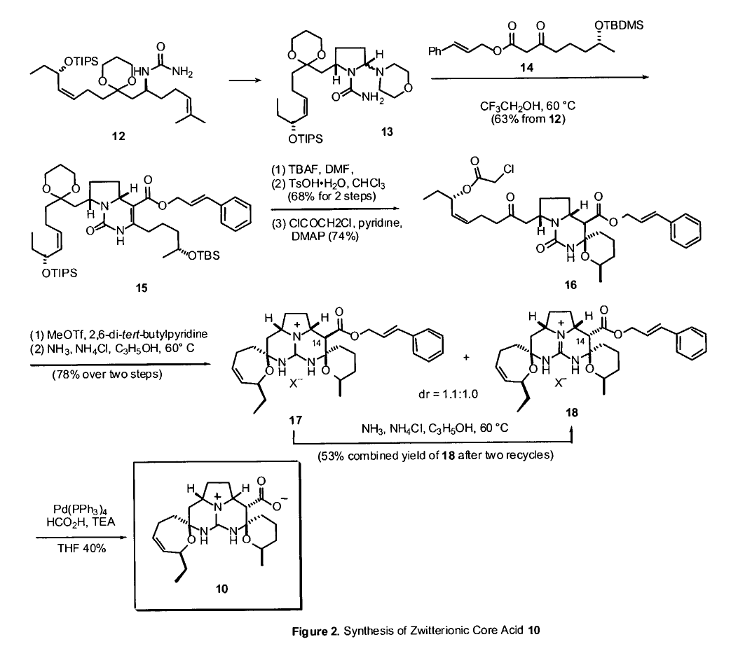

FIG. 3 depicts a X-ray Model of Zwitterionic crambescidin core Acid (compound 10) (Methanol solvate).

FIG. 4 depicts synthesis of side chain analog Crambescidin 431, using Mitsunobu coupling as described in Example 2, infra.

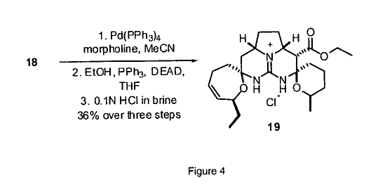

FIG. 5 depicts syntheses of side chain analogs of Crambescidins 657 and 800, as described in Examples 5 and 6, infra.

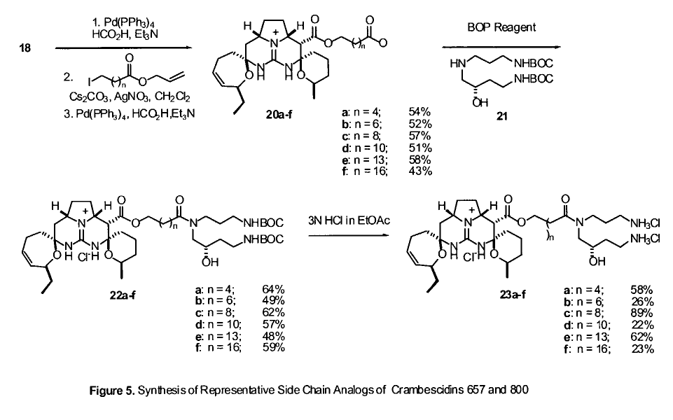

FIG. 6 depicts cytotoxicity profiles of (A), Crambescidin 657 analogs; and (B), Crambescidin 800 analogs, as described in Example 7, infra.

### DETAILED DESCRIPTION OF THE INVENTION

The invention provides methods for the synthesis, isolation, purification, characterization and use of pentacyclic intermediates, including compounds such as a zwitterionic core acid having the carboxylic acid side chain in the natural axial orientation, and their use for preparing therapeutic pentacyclic guanidinium alkaloid analogs.

As used herein the term “natural” means a structure that is identical to a structure that occurs in nature.

As used herein the term “non natural” means a structure that is different from a structure that occurs in nature.

### COMPOSITION OF THE INVENTION

The present invention provides pentacyclic compounds of the general formula:

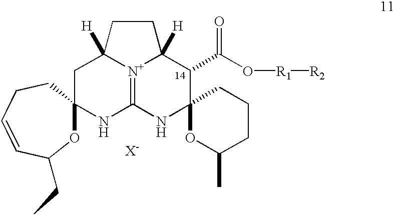

wherein X is any pharmaceutically acceptable counterion; R₁ and R₂ are groups as described below:

The compounds of Formula 11 include pharmaceutically acceptable salts with organic and inorganic acids, organic and inorganic bases, or may be in the form of a zwitterion.

In one embodiment, the invention provides pentacyclic compounds of the Formula 11, where R1 is a hydrocarbyl group with from 1 to 20 atoms or is absent; and where R2 is absent or is selected from the group consisting of H, alkyl, aryl, heteroaryl, carboxy, carboxylate anion, phosphonate, phosphate, sulphonate, sulphate, borate, boronate and amine.

In another embodiment, the invention provides pentacyclic compounds of formula 11, where the hydrocarbyl group of R1 is selected from the group consisting of saturated, unsaturated, cyclic, acyclic, straight, branched chiral and achiral hydrocarbyl groups.

In another embodiment, the invention provides pentacyclic compounds of formula 11, where one or more carbon in R1 is replaced with one or more elements selected from O, S, or NR3, wherein R3 is alkyl, cycloalkyl or acyl.

In another embodiment, the invention provides pentacyclic compounds of formula 11, where one or more carbons in R1 is substituted with any alkyl or aryl group, including but not limited to aralkyl, heteroalkyl or a heteroaralkyl group.

In another embodiment, the invention provides pentacyclic compounds of formula 11, where the R1 is further substituted with one or more groups selected from the group consisting of halo, nitro, cyano, trifluoromethyl, hydroxy, thio, methylthio, carboxy, carboalkoxy, amino, any substituted amino, including but not limited to acylamino, aminoalkylamino, guanidine groups.

In another embodiment, the invention provides pentacyclic compounds of formula 11, where any one carbon in R¹ is substituted with an aryl or heteroaryl group having from 6 to 14 carbon atoms.

In another embodiment, the invention provides pentacyclic compounds of formula 11, where the aryl or heteroaryl group is selected from the group consisting of phenyl, naphthyl, biphenylyl, furyl, thienyl, pyrrolyl, imidazolyl, benzofuranyl, benzoxazolyl, benzothiazolyl, thianaphthyl and indolyl.

In another embodiment, the invention provides pentacyclic compounds of claim 1, where the alkyl, aryl, or heteroaryl group is substituted with one or more groups selected from the group consisting of halo, nitro, cyano, trifluoromethyl, hydroxy, thio, methylthio, amino, substituted amino, acylamino and guanidino.

In another embodiment, the invention provides pentacyclic compounds of formula 11, where R2 is an amine. In additional embodiments of the pentacyclic compound of formula 11, the amine is NR₄R₅ or caroxamide-C0-NR₄R₅, where R₄ and R₅ are selected from the group consisting of H, alkyl, aralkyl, carboxyalkyl, amino-iminomethyl, hydroxyalkyl, acyl, aminoalkyl. The carboxyalkyl may be substituted at the alkyl carbon by hydroxyalkyl, alkyl, thioalkyl, aminoalkyl, carboxyalkyl, amidoalkyl, guanidinoalkyl, aminoalkylaminoalkyl, hydroxyalkylaminoalkyl and acyl derivatives thereof. The hydroxyalkyl, acyl, or aminoalkyl may substituted with one or more hydroxy groups.

In another embodiment, the invention provides pentacyclic compounds of formula 11, wherein R₄ and R₅ combine to form a cyclic ring. The cyclic ring may be substituted with a heteroatom; where the heteroatom is O(morpholino). Additionally, the heteroatom can be NR₈ where R₈ is selected from the group consisting of H(piperazino), alklyl (alkyl piperazino), carboxyalkyl, hydroxyalkyl and aminoalkyl.

In another embodiment, the invention provides pentacyclic compounds of formula 11, where the amino-iminomethyl has the formula —C═N—R₆(—NR₇), and R₆ and R₇ are selected from H and alkyl, or are bridged with 2 to 4 carbons to form a cyclic structure, provided that when R₂ is carboxylate anion or carboxamine.

In a specific embodiment, the invention provides pentacyclic compounds of formula 11, where R₁ is —CH₂—CH═CH—, R₂ is phenyl.

In another embodiment, the invention provides pentacyclic compounds of formula 11, where R1 is H or alkyl; and R2 is absent.

In another embodiment, the invention provides pentacyclic compounds of formula 11, where in R1, the alkyl group is decyl.

In another embodiment, the invention provides pentacyclic compounds of formula 11, where R1 is —(CH2-CH2-O—)n, R2 is alkyl; and n=1-20.

In another embodiment, the invention provides pentacyclic compounds of formula 11, where R1 is —(CH2-CH2-O—)n, R2 is NR4R5; and where n=1-20; and R4 and R5 are H.

In another embodiment, the invention provides pentacyclic compounds of formula 11, where R1 is a carboxyalkyl [CH2-(CH2)n] group, n=1-20; and R2 is an alkyl or allyl group.

The invention further provides the pentacyclic compounds of formula 11, where R₁ is —CH₂—CH═CH—, R₂ is phenyl. (compound 18) of formula:

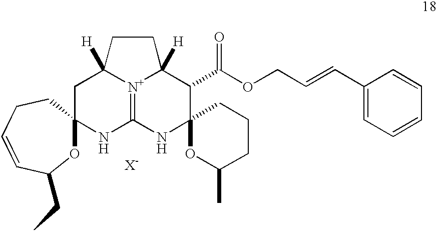

In another embodiment, the invention provides compounds of formula 11, where R1 is H or alkyl; and R2 is absent.

In another embodiment, the invention provides compounds of formula 11, where R1 is (CH2-CH2-O—)n; and R2 is alkyl.

In a preferred embodiment, the invention provides a pentacyclic zwitterionic core compound having the carboxylate side chain at C14 in natural axial orientation (compound 10) of formula:

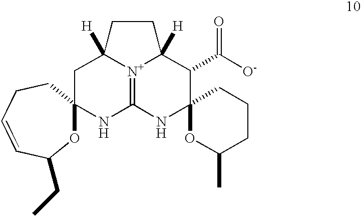

### METHODS OF THE INVENTION

The invention includes methods for preparing the compound having the formula 11:

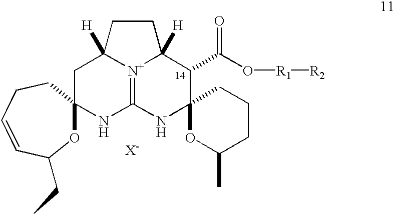

where X=any pharmaceutically acceptable counterion; R1 and R2 are described above, by:

(a) reacting a compound of formula:

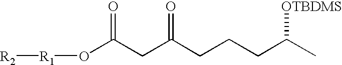

wherein, TBDMS is an alcohol protecting group with a compound of formula:

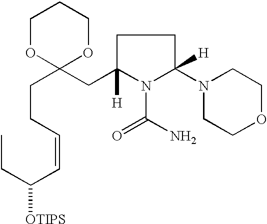

wherein TIPS is an alcohol protecting group to produce a compound of the formula:

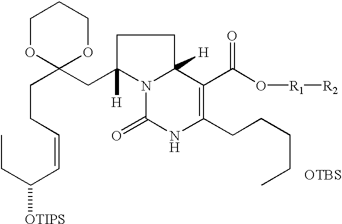

wherein TBPS and TIPS are alcohol protecting groups;

(b) which is converted by deprotection, incorporation of ammonia, and cyclization to pentacyclic compound having the ester side chain in natural axial orientation of the formula:

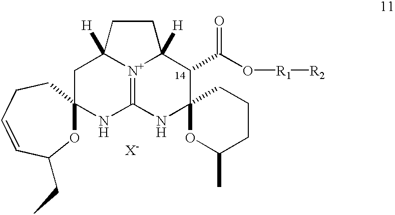

where X=any pharmaceutically acceptable counterion.

In a specific embodiment, invention provides a method for synthesizing a compound of formula 11, where R₁ is —CH₂—CH═CH—, R₂ is phenyl (compound 18), by

(a) reacting a compound of formula:

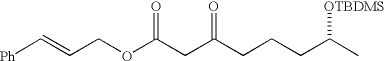

wherein, TBDMS is an alcohol protecting group with a compound of formula:

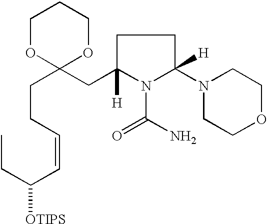

wherein TIPS is an alcohol protecting group;

to produce a compound of the formula:

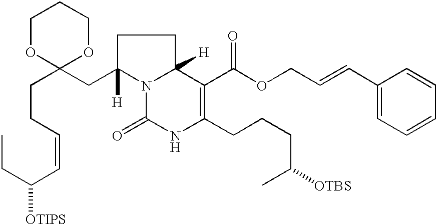

wherein TBPS and TIPS are alcohol protecting groups;

(b) which is converted by deprotection, incorporation of ammonia, and cyclization to pentacyclic compound having the ester side chain in natural axial orientation of the formula:

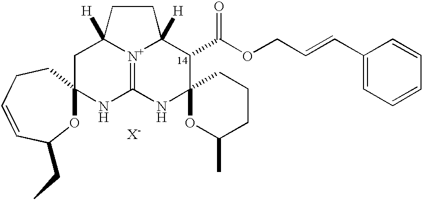

where X is any pharmaceutically acceptable counterion.

In another embodiment, the invention provides methods for synthesizing the pentacyclic zwitterionic compound of formula:

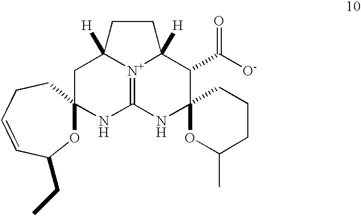

Which comprises palladium mediated deprotection of ester side chain compound of formula 11:

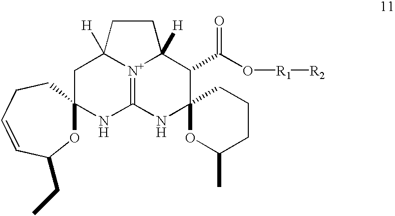

to produce the pentacyclic zwitterionic compound 10.

The invention further provides methods for using the pentacyclic zwitterionic core acid (compound 10) for the synthesis of non-natural side chain analogs (Formula 11) of the Crambescidin/Ptilomycalin alkaloids.

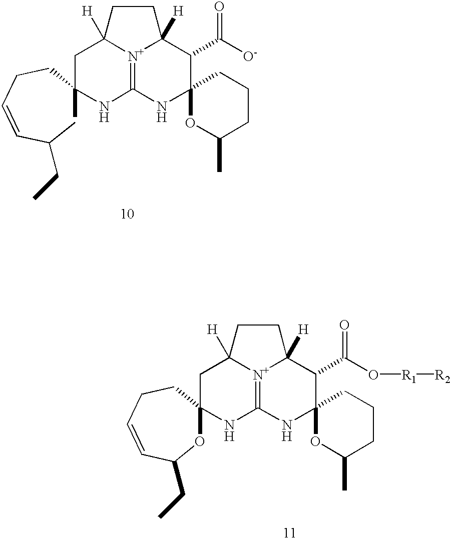

where, R₁ and R₂ are as described above.

The application further describes methods for coupling the zwitterionic core compound (compound 10) with side chain fragments R₁—R₂ by reacting the zwitterionic core acid (compound 10) with an alcohol (HO—R₁—R₂) or an iodide (I—R₁—R₂) to provide pentacyclic guanidinium alkaloid analogs covered by Formula 11.

In one embodiment, Mitsunobu coupling of the zwitterionic compound 10 with ethanol is used to obtain Crambescidin 431 (compound 19)

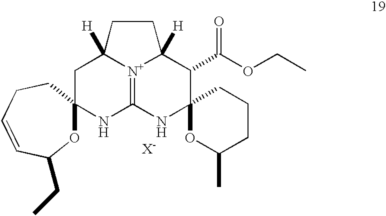

where, X=any pharmaceutically acceptable counterion.

In another embodiment, coupling of zwitterionic compound 10 with an alkyl or alkenyl hydroxy alkanoate provides an ester side chain compound having the formula:

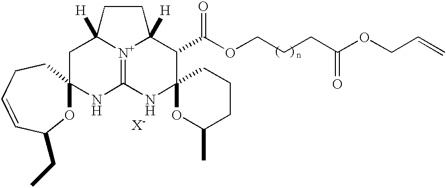

where, n=1-20.

X=any pharmaceutically acceptable carrier.

In a specific embodiment, allyl 16-hydoxyhexadecanoate is used to obtain allyl ester analogs (e.g., compound 27).

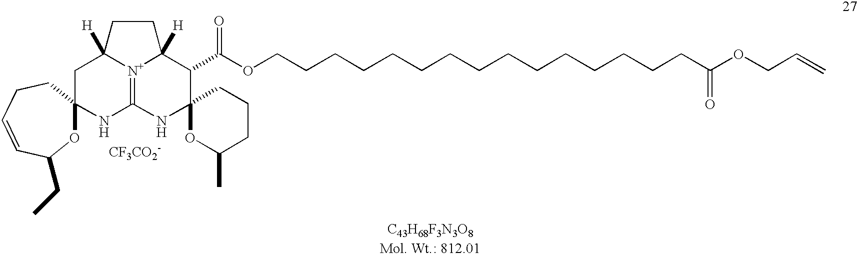

In another embodiment, coupling of zwitterionic compound 10 with a hydroxy oxaalkane such as HO—(CH₂—CH₂—O)₂—CH₂—CH₃ is used to obtain compound 9.

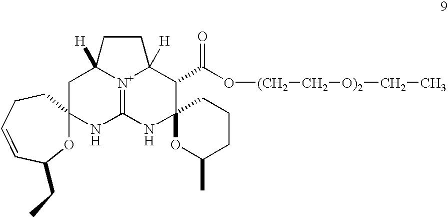

In another embodiment, coupling of compound 10 with ω-iodoesters was used to obtain Crambescidin 657 analogs of formula:

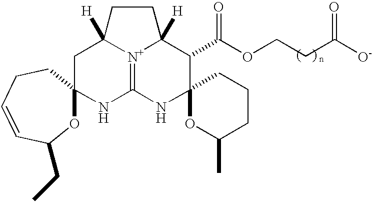

where, n=1-20.

In another embodiment, coupling of above-obtained zwitterionic Crambescidin 657 analogs (C14 carboxyalkyl esters) with

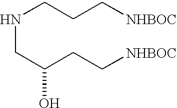

and removal of the BOC blocking group provides Crambescidin 800 analogs.

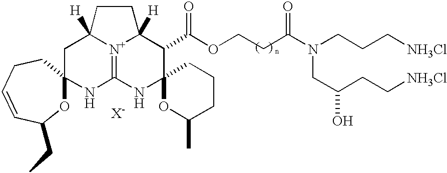

where, n=1-20, and X=any pharmaceutically acceptable counter ion.

In another embodiment coupling of the pentacyclic zwitterionic core acid (compound 10) with an amino alcohol having structure HO(CH2)n-NH2 provides an amino alkyl ester, which is treated with an alkyl-S—C—NH2(═NH) to produce the pentacyclic compound of structure 11, wherein R1-R2 comprises a guanidino alkyloxy group of structure —(CH₂)n-NH═C—NH₂.

Protecting groups and strategies for synthesis of organic compounds are well known in the art (Protective Groups in Organic Synthesis, 2^{nd} Ed. T. W. Greene, P. G. M. Wuts, J. Wiley and Sons, Inc. New York, 1991).

Carboxylic acid protecting groups may be chosen from the following group including, but not limited to, esters and amides. Alcohol protecting groups may be chosen from the following groups including, but not limited to, ether groups, silyl protecting groups, such as TIPS, TBDMS, SEM, THP, TES, TMS, or ester groups, such as acetates, benzoates, and mesitoates.

Carbonyl protecting groups may be chosen from the following groups including, but not limited to, ethers, cyclic or acyclic acetals, ketals, thioketals and thioacetals.

Amine protecting groups may be chosen from the following groups including, but not limited to, N-alkyls, such as benzyl, methyl, N-Silyl groups, N-acyl groups and N-carbamates.

Pharmaceutically acceptable counterions may be chosen from the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate.

Enantiomers of the compounds are important variations of this invention. In addition, the invention encompasses the novel chemical intermediates and methods for the preparation of the compounds.

The compounds of the invention may be used in therapy as antiviral, antifungal and/or as antitumor agents. The compounds of the invention may be further used as Ca²⁺ channel blocking agents. The invention includes the pharmacologically acceptable counterions of the compounds and pharmaceutical preparations for oral, transdermal and parenteral administration of the compounds. For such uses, the compounds are administered intravenously, intramuscularly, topically, transdermally by means of skin patches, by transmucosal application, including nasal, sublingual, buccal, vaginal and rectal or orally to man or other animals. The compositions can be presented for administration to humans and animals in a variety of dosage forms which include, but are not limited to, liquid solutions or suspensions, tablets, pills, powders, suppositories, polymeric microcapsules or microvesicles, liposomes, and injectable or infusible solutions, granules, sterile parenteral solutions or suspensions, oral solutions or suspensions, oil in water and water in oil emulsions containing suitable quantities of the compound, suppositories and in fluid suspensions or solutions. The preferred form depends upon the mode of administration and the therapeutic application.

For oral administration, either solid or fluid unit dosage forms can be prepared. For preparing solid compositions such as tablets, the compound can be mixed with conventional ingredients e.g. talc, magnesium stearate, dicalcium phosphate, magnesium aluminum silicate, calcium sulfate, starch, lactose, acacia, methylcellulose, and functionally similar materials as pharmaceutical diluents or carriers. Capsules are prepared by mixing the compound with an inert pharmaceutical diluent and filling the mixture into a hard gelatin capsule of suitable size. Soft gelatin capsules are prepared by machine encapsulation of a slurry of the compound with vegetable oil, light liquid petrolatum or other inert oil.

Dosage forms for oral administration include syrups, elixirs, and suspensions. The forms can be dissolved in an aqueous vehicle along with sugar, aromatic flavoring agents and preservatives to form a syrup. Suspensions can be prepared with an aqueous vehicle with the aid of a suspending agent for example acacia, tragacanth, methylcellulose and the like.

For parenteral administration, fluid unit dosage forms can be prepared utilizing the compound and a sterile vehicle. In preparing solutions the compound can be dissolved in the vehicle for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The dry lyophilized powder can then be sealed in the vial and reconstituted prior to use.

The most effective mode of administration and dosage regimen for the molecules of the present invention depends upon the severity and course of the disease, the subject's health and response to treatment and the judgment of the treating physician. Accordingly, the dosages of the molecules should be titrated to the individual subject.

Adjustments in the dosage regimens and/or modes of administration may be made to optimize the antiviral, antifungal or antitumor efficacy of the compounds of the invention.

Efficacy of the compounds of the invention in therapy may be assessed using known methods. For example, efficacy of the compounds as anti-tumor agents may be assessed by tumor biopsy or non-invasive procedures to determine tumor growth inhibition. Similarly, efficacy of the compounds as anti-viral or anti-fungal agents may be determined using standard protocols such as assays to detect decreases in numbers of viral particles or fungal cells, or in the numbers of virally or fungally infected cells.

The following examples are presented to illustrate the present invention and to assist one of ordinary skill in making and using the same. The examples are not intended in any way to otherwise limit the scope of the invention.

### EXAMPLES

Experimental details were generally as described (U.S. Ser. No. 10/018,630 and PCT/US00/18395), incorporated by reference herein. IR spectra were obtained using an Applied Systems ReactIR 1000.

### Example 1

This example describes a method for synthesis, isolation and characterization of Zwitterionic core acid (compound 10).

a. (2′E)-3′-phenyl-2′-propenyl (7R)-7-(tert-butyldimethylsilyl)-3-oxo-octanoate (14)

A solution of cinnamyl alcohol (62 g, 463 mmol), methyl (7R)-7-(tert-butyldimethylsilyl)-3-oxo-octanoate (23 g, 76 mmol), 4-dimethylaminopyridine (9.3 g, 76 mmol) and acetonitrile (1.0 L) was heated to 92° C. (external temperature) and the volume of the solution was kept constant by continuous addition of acetonitrile.

After 10 hours, the solution was concentrated to an oil, which was maintained at 92° C. for an additional 10 hours. The resultant oil was allowed to cool to room temperature and was purified by silica gel chromatography (10/1 hexanes/ethyl acetate) to give 22.6 g (55.8 mmol, 74%) of pure (2′E)-3′-phenyl-2′-propenyl (7R)-7-(tert-butyldimethylsilyl)-3-oxo-octanoate (compound 14) as a 0.1:1 mixture of enol:keto tautomers: ¹H NMR (500 MHz, DMSO) δ11.95 (br s, 0.1H), 7.43-7.46 (m, 2H), 7.32-7.35 (m, 2H), 7.25-7.28 (m, 1H), 6.68 (d, J=16.0 Hz, 1H), 6.31-6.41 (m, 1H), 5.14 (s, 0.1H), 4.77 (d, J=5.3 Hz, 0.3H), 4.73 (d, J=6.1 Hz, 1.6H), 3.73-3.82 (m, 1H), 3.62-3.63 (m, 1.7H), 2.48-2.54 (m, 1.7H), 2.17-2.22 (m, 0.3H), 1.15-1.61 (m, 4H), 1.03-1.06 (m, 3H), 0.82-0.83 (m, 9H), 0.00-0.01 (m, 6H); ¹³C NMR (125 MHz, DMSO) 202.8, 166.6, 135.5, 132.9, 128.3, 128.29, 127.7, 126.14, 126.10, 123.0, 67.6, 64.7, 48.7, 42.1, 38.3, 30.6, 25.8, 23.7, 22.5, 21.9, 19.2, 17.8, −4.3, −4.7 ppm; IR (film) 3061, 3030, 2957, 2934, 2887, 2860, 1745, 1718 cm⁻¹; HRMS (ES) calculated for C₂₃H₃₆O₄SiNa 427.2281, found 427.2278; [α]²⁶₄₀₅ −21.8, [α]²⁶₄₃₅ −18.4, [α]²⁶₅₄₆ −10.5, [α]²⁶₅₇₇ −9.4, [α]²⁶₅₈₉ −8.9 (c 1.0, methylene chloride).

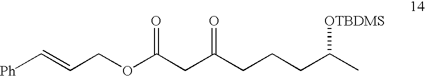

b. (4aR,7S)-4-((2bE)-3b-Phenyl-2b-propenyloxy carbonyl)-1,2,4a,5,6,7-hexahydro-3-[(4S)-(tert-butyldimethylsilyl)pentyl]-7-[(7S,5Z)-2-(1′,3′-dioxan-2′-yl)-7-(triisopropylsilyl) non-5-enyl]-1-oxo-pyrollo[1,2-c]pyrimidine (15)

A solution of osmium tetroxide (4.9 mL, 0.1 M in tert-butanol) was added to a solution of (6S, 11Z, 13S)-8-(1′,3′-Dioxan-2′-yl)-2-methyl-13-triisopropylsiloxy-6-ureidopentadeca-2,11-diene (compound 12) (3.4 g, 6.5 mmol), N-methylmorpholine-N-oxide (3.1 g, 23 mmol), tetrahydrofuran (160 mL) and water (18 mL). After 1.5 hours, Florisil (19.5 g), sodium bisulfite (19.5 g), and ethyl acetate (325 mL) were added and the reaction mixture was stirred vigorously. After 0.8 hours, the reaction mixture was filtered and concentrated in vacuo to provide the desired diol as a colorless oil, which was carried forward without further purification.

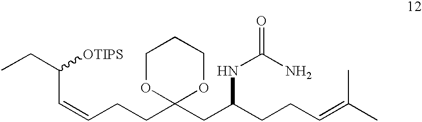

Lead(IV)acetate (3.5 g, 7.9 mmol) was added portionwise over 40 minutes to a solution of the above diol and toluene (390 mL). Following complete consumption of the starting material by TLC analysis (1.25 hours), the reaction mixture was filtered through a plug of Celite. Morpholine (2.51 mL, 28.5 mmol) and glacial acetic acid (1.50 mL, 26 mmol) were added sequentially to the resultant filtrate, which was then concentrated in vacuo (60° C., 45 minutes) to provide crude aminal compound 13 as a heterogeneous mixture. This material was used without further purification.

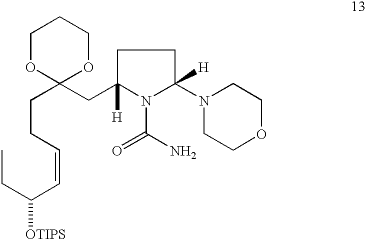

A solution of crude aminal compound 13 prepared above, (2′E)-3′-phenyl-2′-propenyl (7R)-7-(tert-butyldimethylsilyl)-3-oxo-octanoate (compound 14)(8.84 g, 21.9 mmol), obtained above under Example 1a, and 2,2,2-trifluoroethanol (6.51 mL), was maintained at 60° C. for 56 hours. The reaction mixture was then allowed to cool to room temperature, diluted with ether (130 mL), and washed with 50% aqueous ammonium chloride (30 mL). The organic layer was dried (magnesium sulfate), filtered, and concentrated in vacuo. Purification by silica gel chromatography (10/1 hexanes/ethyl acetate; 7/1 hexanes/ethyl acetate; 3/1 hexanes/ethyl acetate) provided 4.3 g of recovered (2′E)-3′-phenyl-2′-propenyl (7R)-7-(tert-butyldimethylsilyl)-3-oxo-octanoate (compound 14) and 3.6 g (4.2 mmol, 63%) of a ˜6:1 mixture of (4aR,7S)-4-((2bE)-3b-Phenyl-2b-propenyloxy carbonyl)-1,2,4a,5,6,7-hexahydro-3-[(4S)-(tert-butyldimethylsilyl)pentyl]-7-[(7S,5Z)-2-(1′,3′-dioxan-2′-yl)-7-(triisopropylsilyl)non-5-enyl]-1-oxo-pyrollo[1,2-c]pyrimidine (compound 15) and its C13 epimer, respectively, which was carried forward without further separation.

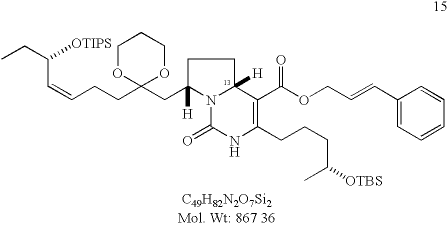

For characterization purposes, a 25 mg sample of this mixture was separated by HPLC (10/1 hexanes/ethyl acetate; 10/1.5 hexanes/ethyl acetate; Altima 5 μ silica) to give (4aR,7S)-4-((2bE)-3b-Phenyl-2b-propenyloxy carbonyl)- 1,2,4a,5,6,7-hexahydro-3-[(4S)-(tert-butyldimethylsilyl)pentyl]-7-[(7S,5Z)-2-(1′,3′-dioxan-2′-yl)-7-(triisopropylsilyl) non-5-enyl]-1-oxo-pyrollo[1,2-c]pyrimidine (compound 15) as a single diastereomer. ¹H NMR (500 MHz, CDCl₃) δ7.38-7.40 (m, 2H), 7.31-7.34 (m, 2H), 7.26-7.28 (m, 1H), 6.66 (d, J=15.9 Hz, 1H), 6.27-6.33 (m, 2H), 5.31-5.37 (m, 2H), 4.76-4.80 (m, 2H), 4.51 (dd, J=7.1, 5.6 Hz, 1H), 4.26 (dd, J=11.1, 4.8 Hz, 1H), 4.11-4.15 (m, 1H), 3.95-4.02 (m, 1H), 3.76-3.96 (m, 4H), 2.55-2.64 (m, 2H), 2.48-2.55 (m, 1H), 2.40 (d, J=13.0 Hz, 1H), 2.23-2.32, (m, 1H), 2.17 (dd, J=12.8, 6.1 Hz, 1H), 1.95-2.05 (m, 1H), 1.73-1.95 (m, 3H), 1.55-1.73 (m, 4H), 1.39-1.52 (m, 3H), 1.24-1.33 (m, 3H), 109 (d, J=6.1 Hz, 3H), 1.04 (s, 21H), 0.86-0.88 (m, 12H), 0.03 (s, 3H), 0.03 (s, 3H); ¹³C NMR (125 MHz, CDCl₃) 165.3, 151.31, 151.29, 135.9, 134.2, 134.1, 128.4, 128.0, 127.9, 126.4, 123.1, 101.8, 99.1, 69.9, 68.8, 64.8, 59.44, 59.41, 57.8, 52.9, 39.2, 37.6, 34.5, 32.0, 31.7, 30.6, 29.2, 26.1, 25.5, 24.5, 23.8, 22.0, 18.34, 18.32, 12.6, 9.6, −4.1, −4.3 ppm; IR (film) 3212, 3088, 2941, 2864, 1679, 1625, 1108, 1069 cm⁻¹; HRMS: (FAB) calculated for C₄₉H₈₂N₂O₇Si₂ (M+Na⁺) 889.5558, found 889.5558; [α]²⁶₄₀₅ −16.6, [α]²⁶₄₃₅ −12.4, [α]²⁶ ₅₄₆ −5.0, [α]²⁶₅₇₇ −5.0, [α]²⁶₅₈₉ −4.7 (c0.3, methylene chloride)

c. (3R,4R,4aR,6′R,7S)-4-((2bE)-3b-phenyl-2b-propenyloxy carbonyl)-1,2,4a5,6,7-hexa-hydro-1-oxo-7-[(7S,5Z)-7-hydroxy-2-oxo-5-nonenyl]-pyrrolo[1,2-c]pyrmidine-3-spiro-6′-(2′-methyl)-3′,4′,5′,6′-tetrahydro-2H-pyran (compound 25)

A solution of tetra-butylammonium fluoride (13 mL, 1 M in tetrahydrofuran) was added to a solution of (4aR,7S)-4-((2bE)-3b-Phenyl-2b-propenyloxy carbonyl)-1,2,4a,5,6,7-hexahydro-3-[(4S)-(tert-butyldimethylsilyl)pentyl]-7-[(7S,5Z)-2-(1′,3′-dioxan-2′-yl)-7-(triisopropylsilyl) non-5-enyl]-1-oxo-pyrollo[1,2-c]pyrimidine and its C13 epimer (compound 15) (2.3 g, 2.7 mmol), obtained under Example 1b, and dimethylformamide (67 mL). The reaction was maintained at room temperature for 5.5 hours, diluted by the addition of ether (350 mL) and washed with aqueous lithium chloride (50 mL, 50% saturated). The aqueous phase was extracted with ethyl acetate (30 mL) and the resultant organic phases washed with water (20 mL). The organic extracts were combined; washed with water (50 mL) and brine (2×50 mL); dried (magnesium sulfate); filtered and concentrated in vacuo. The resultant diol was used without further purification in the next reaction.

Solid p-toluenesulfonic acid monohydrate (0.50 g, 2.7 mmol) was added to a solution of the above diol and chloroform (390 mL). The reaction was maintained at room temperature for 45 minutes, then partitioned from saturated aqueous sodium bicarbonate (40 mL). The organic layer was washed with brine (30 mL), dried (magnesium sulfate), filtered, and concentrated in vacuo to a yellow oil. Purification of this oil by silica gel chromatography (ethyl acetate) gave 0.97 g (1.8 mmol, 67%) of (3R,4R,4aR,6′R,7S)-4-((2bE)-3b-phenyl-2b-propenyloxy carbonyl)-1,2,4a,5,6,7-hexa-hydro-1-oxo-7-[(7S,5Z)-7-hydroxy-2-oxo-5-nonenyl]-pyrrolo[1,2-c]pyrmidine-3-spiro-6′-(2′-methyl)-3′,4′,5′,6′-tetrahydro-2H-pyran (compound 25) as a ˜7:1 mixture of C10 epimers. This mixture of stereoisomers was not separated, but directly used in the next step.

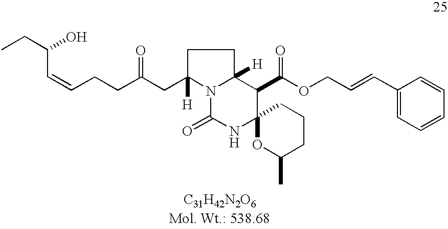

A pure sample of (3R,4R,4aR,6′R,7S)-4-((2bE)-3b-phenyl-2b-propenyloxy carbonyl)-1,2,4a,5,6,7-hexa-hydro-1-oxo-7-[(7S,5Z)-7-hydroxy-2-oxo-5-nonenyl]-pyrrolo[1,2-c]pyrmidine-3-spiro-6′-(2′-methyl)-3′,4′,5′,6′-tetrahydro-2H-pyran (compound 25) was obtained by exhaustive silica gel chromatography (ethyl acetate; 100/1 ethyl acetate/iso-propanol): ¹H NMR (500 MHz, CDCl₃) δ7.37-7.39 (m, 2H), 7.32-7.35 (m, 2H), 7.26-7.29 (m, 1H), 6.69 (d, J=15.8 Hz, 1H), 6.26 (dt, J=15.8, 6.2 Hz, 1H), 5.62 (s, 1H), 5.34-5.43 (m, 2H), 4.76-4.82 (m, 2H), 4.38 (q, J=7.1 Hz, 1 H), 4.19 (td, J=7.9, 0.5 Hz, 1 H), 4.05 (td, J=11.1, 5.0 Hz, 1 H), 3.76-3.78 (m, 1H), 3.38 (d, J=16.6 Hz, 1H), 2.80 (br s, 1H), 2.41-2.65 (m, 3H), 2.32 (d, J=11.2 Hz, 1H), 2.23 (dd, J=16.8, 9.72 Hz, 1 H), 2.05-2.19 (m, 4H), 1.44-1.74 (m, 9H), 1.02-1.09 (m, 4H), 0.89 (t, J=7.3 Hz, 3 H); ¹³C NMR (125 MHZ, CDCl₃) 208.4, 168.2, 152.6, 135.8, 134.5, 133.9, 129.67, 129.57, 128.5, 128.0, 126.4, 122.5, 82.1, 68.4, 66.2, 65.5, 55.1, 54.1, 53.3, 46.3, 42.8, 32.4, 30.1, 29.7, 29.4, 22.2, 21.8, 19.0, 10.0 ppm; IR (film) 3389, 3304, 3231, 2968, 2934, 2880, 1729, 1714, 1652, 1475 cm⁻¹; HRMS: (ES) calcd for C₃₁H₄₂N₂O₆Na 561.2941, found 561.2922. [α]²⁶₄₀₅ +108.7, [α]²⁶₄₃₅ +86.6, [α]²⁶₅₄₆ +46.5, [α]²⁶₅₇₇ +40.7, [α]²⁶₅₈₉ +38.6 (c 1.0, methylene chloride). Anal. Calculated for C₃₁H₄₂N₂O₆: C, 69.12; H, 7.86; N, 5.20. Found: C, 69.34; H, 8.03; N, 5.10.

d. (3R,4R,4aR,6′R,7S)-4-((2bE)-3b-phenyl-2b-propenyloxycarbonyl)-1,2,4a,5,6,7-hexahydro-1-oxo-7-[(7S,5Z)-7-chloroacetoxy-2-oxo-5-nonenyl]-pyrollo-[1,2-c]-pyrimidine-3-spiro-6′-(2′-methyl)-3′,4′,5′,6′-tetrahydro-2H-pyran (compound 16).

α-Chloroacetyl chloride (0.47 mL, 6.0 mmol) was added dropwise over 10 minutes to a 0° C. solution of (3R,4R,4aR,6′R,7S)-4-((2bE)-3b-phenyl-2b-propenyloxy carbonyl)-1,2,4a,5,6,7-hexa-hydro-1-oxo-7-[(7S,5Z)-7-hydroxy-2-oxo-5-nonenyl]-pyrrolo[1,2-c]pyrmidine-3-spiro-6′-(2′-methyl)-3′,4′,5′,6′-tetrahydro-2H-pyran (compound 25) (0.64 g, 1.2 mmol containing ˜12% of C4a S epimer), obtained under Example 1c, 4-dimethylaminopyridine (0.064 g, 0.52 mmol), pyridine (1.9 mL, 24 mmol) and methylene chloride (51 mL). After 30 minutes at 0° C., the reaction was allowed to warm to room temperature over 2 hours. This was diluted with ether (200 mL); washed with 1 N sodium hydroxide (40 mL), copper sulfate (2×40 mL) and brine (20 mL); dried (magnesium sulfate); filtered; and concentrated in vacuo. Purification of the resultant viscous oil by silica gel chromatography (1/1 hexanes/ethyl acetate) gave 0.55 g (0.89 mmol, 74%) of (3R,4R,4aR,6′R,7S)-4-((2bE)-3b-phenyl-2b-propenyloxycarbonyl)-1,2,4a,5,6,7-hexahydro-1-oxo-7-[(7S,5Z)-7-chloroacetoxy-2-oxo-5-nonenyl]-pyrollo-[1,2-c]-pyrimidine-3-spiro-6′-(2′-methyl)-3′,4′,5′,6′-tetrahydro-2H-pyran (compound 16) as a single isomer: ¹H NMR (500 MHz, CDCl₃) δ7.38-7.39 (m, 2H), 7.32-7.35 (m, 2H), 7.26-7.28 (m, 1H), 6.67 (d, J=15.9 Hz, 1H), 6.26 (dt, J=15.9, 6.5 Hz, 1H), 5.49-5.56 (m, 3H), 5.27-5.32 (m, 1H), 4.77-4.85 (m, 2H), 4.34 (td, J=9.1, 3.1 Hz, 1H), 4.06 (td, J=11.1, 5.0 Hz, 1H), 4.02 (s, 2H), 3.77-3.82 (m, 1H), 3.36 (dd, J=16.8, 2.8 Hz, 1H), 2.43-2.50 (m, 3H), 2.25-2.38 (m, 3H), 2.12-2.21 (m, 2H), 2.03-2.08 (m, 1H), 1.67-1.79 (m, 3H), 1.47-1.64 (m, 3H), 1.25 (br s, 2H), 1.03-1.11 (m, 4H), 0.90 (t, J=7.46 Hz, 3H); ¹³C (125 MHz, CDCl₃) 207.6, 168.3, 166.3, 152.5, 135.8, 134.5, 132.9, 128.4, 128.01, 127.6, 126.4, 122.5, 82.2, 73.8, 66.2, 65.5, 55.2, 54.1, 53.3, 46.5, 42.5, 41.3, 32.42, 32.38, 29.7, 29.3, 27.7, 22.1, 21.9, 19.0, 9.6 ppm; IR (film) 3304, 3223, 2968, 2934, 2883, 1729, 1652, 753 cm⁻¹; HRMS: (ES) calcd for C₃₃H₄₃ClN₂O₇Na (M+Na) 637.2656, found 637.2663. [α]²⁶₄₀₅ +110.7, [α]²⁶₄₃₅ +88.0, [α]²⁶₅₄₆ +46.7, [α]²⁶₅₇₇ +41.2, [α]²⁶₅₈₉ +39.6 (c 0.3, methylene chloride). Anal. Calculated For C₃₃H₄₃ClN₂O₇: C, 64.43; H, 7.05; N, 4.35. Found: C, 64.22; H, 7.08; N, 4.60.

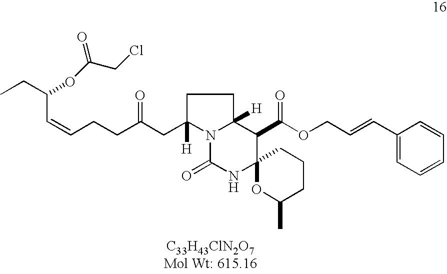

e. Synthesis of Pentacycles (compounds 17 and 18). A solution of (3R,4R,4aR,6′R,7S)-4-((2bE)-3b-phenyl-2b-propenyloxycarbonyl)-1,2,4a,5,6,7-hexahydro-1 -oxo-7-[(7S,5Z)-7-chloroacetoxy-2-oxo-5-nonenyl]-pyrollo-[1,2-c]-pyrimidine-3-spiro-6′-(2′-methyl)-3′,4′,5′,6′-tetrahydro-2H-pyran (compound 16) (0.29 g, 0.46 mmol), obtained under Example 1d, methyl triflate (1.4 mL, 9.1 mmol), 2,6-di-tert-butylpyridine (0.50 mL, 2.3 mmol) and methylene chloride (23 mL) was maintained at room temperature for 9 hours. This solution was diluted with ether (150 mL) and washed with 1 N sodium hydroxide (2×15 mL) and brine (15 mL). The organic layer was dried (magnesium sulfate), filtered and concentrated to give the intermediate methyl pseudourea (compound 26), which was used without further purification.

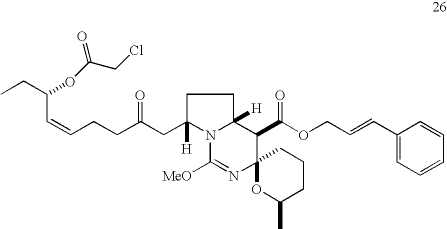

Ammonia was bubbled through a solution of the methyl pseudourea (compound 26) generated above, ammonium chloride (0.055 g, 1.0 mmol), and allyl alcohol (5 mL) for 20 minutes at room temperature. The reaction vessel then was sealed and heated to 60° C. After 20 minutes, the pressure varied, depending on reaction scale, from 18-25 psi. If the pressure was higher than 18 psi, the reaction vessel was vented at this stage to adjust the pressure to 18 psi. The sealed reaction vessel was then maintained at 60° C. and 18 psi for 36 hours. The reaction then was cooled to room temperature and Celite (1 g) was added. This mixture was concentrated in vacuo to give a fine powder. Purification of this powder by silica gel MPLC (100/0.3/0.1 chloroform/iso-propanol/triflouroacetic acid; 100/0.6/0.1 chloroform/iso-propanol/triflouroacetic acid) provided 0.095 g (0.15 mmol) of compound 18, 0.11 g (0.17 mmol) of compound 17 and 0.021 g (0.033 mmol) of a 1.5:1 mixture of A:B (78% overall yield). Resubjection of compound A and the 1.5:1 mixture of 17:18 to these reaction conditions (twice) provided, upon purification, an additional 0.060 g of pure compound 18 (0.24 mmol total, 53% combined yield of compound 18).

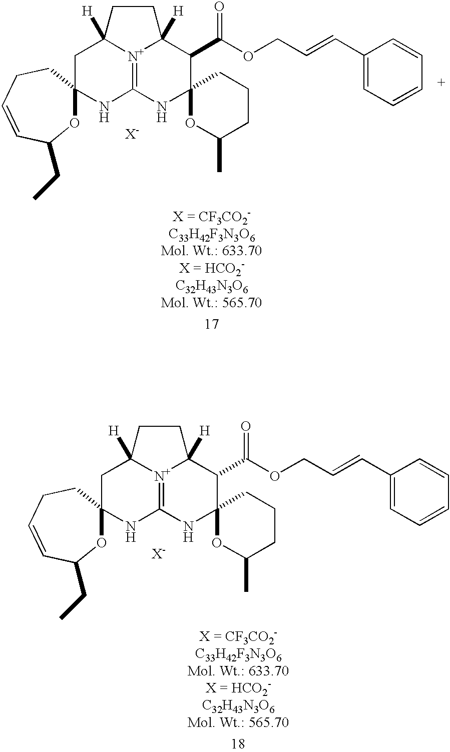

Data for compound 18 trifluoroacetate salt: ¹H NMR (500 MHz, CDCl₃) δ10.02 (br s, 1H), 9.82 (br s, 1H), 7.38-7.42 (m, 2H), 7.32-7.36 (m, 2H), 7.23-7.30 (m, 1H), 6.68 (d, J=15.9 Hz, 1H), 6.26 (dt, J=15.9, 6.6 Hz, 1H), 5.63-5.67 (m, 1H), 5.47 (d, J=10.8 Hz, 1H), 4.73 (d, J=6.6 Hz, 2H), 4.50-4.52 (m, 1H), 4.31 (quint, J=4.9 Hz, 1H), 3.96-4.10 (m, 2H), 2.99 (d, J=4.7 Hz, 1H), 2.42-2.51 (m, 2H), 2.22-2.37 (m, 3H), 2.07-2.21 (m, 2H), 1.50-1.89 (m, 6 H), 1.35-1.48 (m, 2H), 1.15-1.35 (m, 3H), 1.05 (d, J=6.10 Hz, 3H), 0.83 (t, J=7.16 Hz, 3 H); ¹³C (125 MHz, CDCl₃) 168.0, 149.0, 135.9, 135.7, 133.7, 129.8, 128.7, 128.5, 126.7, 121.9, 83.6, 80.7, 71.0, 67.3, 65.9, 53.9, 51.9, 50.1, 36.9, 32.1, 32.0, 30.6, 29.7, 29.1, 26.8, 23.5, 21.5, 18.4, 10.0 ppm; IR (film) 3212, 3088, 2941, 2864, 1679, 1625 cm⁻¹; HRMS: (ES) calcd for C₃₃H₄₂N₃O₆ 520.3175, found 520.3154. [α]²⁶₄₀₅ −17.49, [α]²⁶₄₃₅ −12.24, [α]²⁶₅₄₆ −4.75, [α]²⁶₅₇₇ −4.73, [α]²⁶₅₈₉ −3.74 (c0.3, methylene chloride).

Data for compound 17 trifluoroacetate salt: ¹H NMR (500 MHz, CDCl₃) δ10.48 (s, 1H), 10.17 (s, 1H), 7.38-7.39 (m, 2H), 7.32-7.36 (m, 2 H), 7.26-7.30 (m, 1H), 6.68 (d, J=16.0 Hz, 1H), 6.27 (dt, J=15.9, 6.5 Hz, 1H), 5.62-5.69 (m, 1H), 5.48 (d, J=10.6 Hz, 1H), 4.79-4.88 (m, 2H), 4.42-4.50 (m, 1H), 4.35 (dt, J=11.6, 7.7 Hz, 1H), 4.08-4.17 (m, 1H), 3.77-3.82 (m, 1H), 2.55-2.64 (m, 2H), 2.45 (d, J=11.5 Hz, 1 H), 2.29-2.34 (m, 3H), 2.13-2.16 (m, 2H), 1.94-1.97 (m, 1H), 1.80-1.90 (m, 1H), 1.59-1.77 (m, 4H), 1.50-1.59 (m, 1H), 1.39-1.49 (m, 1H), 1.23-1.34 (m, 2 H), 1.01-1.11 (m, 4 H), 0.82 (t, J=7.2 Hz, 3 H); ¹³C (125 MHz, CDCl₃) 167.4, 147.7, 135.7, 134.9, 133.4, 129.6, 128.5, 128.2, 126.4, 122.0, 83.7, 81.6, 70.8, 67.6, 66.0, 53.7, 53.5, 53.3, 37.1, 36.2, 32.1, 30.9, 30.0, 29.7, 29.3, 23.9, 21.5, 18.2, 10.5 ppm; IR (film) 3231, 3111, 3026, 2968, 2937, 1737, 1675, 1613 cm⁻¹; HRMS: (ES) calcd for C₃₃H₄₂N₃O₆ 520.3175, found 520.3154. [α]²⁶₄₀₅ +6.5, [α]²⁶₄₃₅+16.8, [α]²⁶₅₄₆ +6.7, [α]²⁶₅₇₇ +2.2, [α]²⁶⁵⁸⁹ +1.7 (c 0.3, methylene chloride).

Alternatively, 17 and 18 could be isolated as their formate salts. Isolation by silica gel MPLC (100/0.3/0.2 chloroform/iso-propanol/formic acid; 100/0.6/0.2 chloroform/iso-propanol/formic acid) gave the format salts. Subsequent transformations of α-cinnamyl ester 18 were little effected by the nature of its counter ion.

f. Synthesis of Zwitterionic Core Acid (compound 10). To a vial charged with ester compound 18 formate salt (23.4 mg, 0.0413 mmol), obtained under Example 1e, was added palladium tetrakis(triphenylphosphine) (4.5 mg, 0.0041 mmol) and a solution of triethylammonium formate in tetrahydrofuran (1 M, 0.21 mL). This solution was maintained at room temperature for 14 hours, diluted with freshly distilled acetone (3 mL) and filtered through a fine sintered glass frit. The filtrate was washed with 10 mL of acetone to give 7.9 mg of Zwitterionic Core Acid (compound 10) as a colorless solid (0.020 mmol, 47%) ¹H NMR (500 MHz, 1:1 CDCl₃:CD₃OD—calibrated to CDCl₃) δ 5.38-5.42 (m, 1H), 5.19 (d, J=10.9 Hz, 1H), 3.97-4.02 (m, 1 H), 3.88 (dt, J=8.6, 4.8 Hz, 1H), 3.62-3.70 (m, 1H), 3.42-3.5 (m, 1H), 2.38 (d, J=4.7 Hz, 1H), 2.16 (dd, J=12.7, 4.6 Hz, 1H), 2.08 (br t, J=15.2 Hz, 1H), 1.73-1.96 (m, 5H), 1.63 (dd, J=13.9, 5.8 Hz, 1H), 1.30-1.53 (m, 6H), 1.13-1.28 (m, 3H), 0.92 (q, J=12.0 Hz, 1H), 0.76 (d J=6.2 Hz, 3H) 0.52 (t, J=7.2 Hz, 3H); ¹³C (125 MHz, 1:1 CDCl₃:CD₃OD—calibrated to CDCl₃) 173.1, 148.8, 132.7, 129.8, 83.1, 80.8, 70.2, 65.9, 53.6, 53.1, 52.1, 36.8, 36.0, 31.6, 30.8, 29.6, 28.5, 26.3, 22.9, 20.7 17.9, 9.3 ppm; IR (film) 2961, 2926, 1637 cm⁻¹; HRMS: calculated for C₂₂H₃₄N₃O₄⁺ 404.2549, found 404.2555.

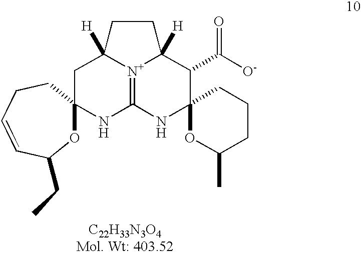

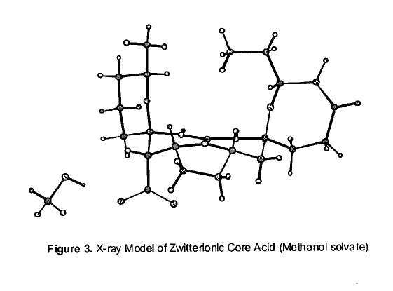

Crystallization of Zwitterionic Core Acid (compound 10) from methanol/methylene chloride by slow evaporation provided X-ray quality crystals of the methanol solvate. The structure of Zwitterionic Core Acid (compound 10) depicted in FIG. 3, is the first X-ray structure of any compound having the natural pentacyclic crambescidin/ptilomycalin A core. Examination of the X-ray structure of Zwitterionic Core Acid (compound 10) shows a significant degree of steric congestion around C14 carboxylate. This steric crowding attenuates the electrophilicity of the carboxylate moiety. Correspondingly, methods to esterify compound 10 under conditions wherein the carboxylate is the nucleophile were developed, specifically, Mitsunobu coupling of compound 10 with alcohols, and direct alkylation of compound 10 with alkyl iodides.

### Example 2

The following Example describes a representative procedure for attaching a side chain (—R₁—R₂) by Mitsunobu Coupling of the crude Core Acid with an alcohol.

Preparation of Crambescidin 431 Trifluoroacetate Salt and Analogs

A solution of ester compound 18 (14 mg, 0.022 mmol), morpholine (9.4 μL, 0.11 mmol), palladium tetrakis(triphenylphosphine) (2.3 mg, 0.0022 mmol) and acetonitrile (2 mL) was maintained at room temperature for 2.5 hours. The reaction then was concentrated in vacuo (2 torr) for 12 hours. The resultant crude core acid (compound 10) was used without further purification.

Diethyl azodicarboxylate (10 μL, 0.052 mmol) was added to a 0° C. solution of this crude core acid, triphenylphosphine (10 mg, 0.038 mmol), ethanol (10 μL, 12 mg, 0.27 mmol) and tetrahydrofuran (2 mL). This solution was maintained at 0° C. for 30 minutes, then allowed to warm to room temperature over 5 hours. The reaction was quenched with water (5 mL), extracted with methylene chloride (25 mL) and concentrated. The residue was azeotroped with benzene (2×25 mL) and the residue was purified by silica gel MPLC (100/0.5/0.1 chloroform/iso-propanol/triflouroacetic acid) to give ethyl ester (compound 19) (4.9 mg, 0.0090 mmol) in 41% yield over two steps. ¹H NMR (500 MHz, CDCl₃) δ10.09 (s, 1H), 9.79 (s, 1H), 5.66 (dd, J=10.3, 7.4 Hz, 1H), 5.48 (d, J=10.9 Hz, 1H), 4.49 (d, J=9.9 Hz, 1H), 4.30 (dt, J=9.45, 5.33 Hz, 1H), 4.12-4.19 (m, 2H), 3.98-4.04 (m, 1 H), 3.85-3.93 (m, 1H), 2.93 (d, J=4 Hz, 1H), 2.55-2.65 (m, 2 H), 2.12-2.40 (m, 5H), 1.60-1.96 (m, 9H), 1.51-1.58 (m, 1H), 1.37-1.50 (m, 2 H) 1.27 (t, J=7.1 Hz, 3H), 1.14-1.23 (m, 1H), 1.05 (d, J=6.1 Hz, 3 H), 0.82 (t, J=7.2 Hz, 3 H); ¹³C (125 MHz, CDCl₃) 168.0, 148.8, 133.4, 129.6, 83.8, 80.7, 70.8, 67.0, 61.3, 54.0, 52.2, 50.1, 36.9, 36.2, 32.1, 31.9, 30.7, 29.3, 26.9, 23.9, 21.7, 18.12, 14.4, 10.4 ppm; IR (film) 3223, 3112, 3102, 2973, 2933, 1733, 1678, 1656, 1610, 720.6 cm⁻¹; MS: (ES) Calculated for C₂₉H₃₉N₃O₄ 432.28, found: 432.10 [α]²⁶₄₀₅ −53.3, [α]²⁶₄₃₅ −49.0, [α]²⁶₅₄₆ −29.6, [α]²⁶₅₇₇ −26.7, [α]²⁶₅₈₉ −27.6 (c 0.19, methylene chloride).

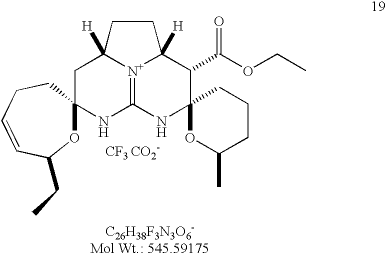

### Example 3

The following Example describes a representative procedure for attaching a side chain (—R₁-R₂) by Mitsunobu Coupling of the purified Core Acid with an Alcohol.

Preparation of Allyl Ester (Compound 27)

To a solution of ester 18 formate salt (16 mg, 0.029 mmol) and tetrahydrofuran (5.7 mL) was added triethylammonium formate (0.14 mL, 1 M in tetrahydrofuran) and palladium tetrakis(triphenylphosphine) (9.4 mg, 0.0086 mmol). This mixture was maintained at room temperature for 4 hours. The reaction then was loaded directly onto a plug of Davisil (3 g) and eluted (100/0/5 methylene chloride/methanol/triethylamine; 100/10/5 methylene chloride/methanol/triethylamine) to give the core acid (compound 10), contaminated with small amounts of Davisil, as a solid.

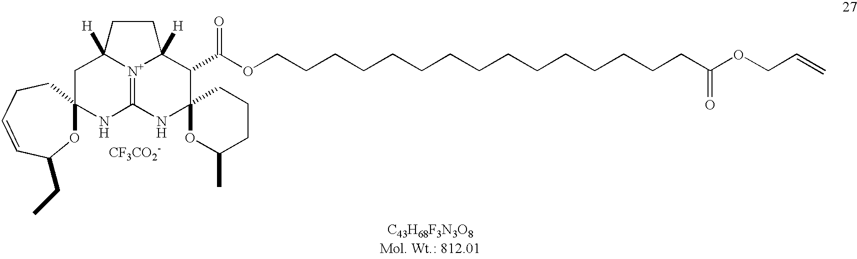

To a 0° C. solution of this solid, triphenylphosphine (23 mg, 0.086 mmol), allyl 16-hydroxy hexadecanoate (26.8 mg, 0.086 mmol), triethylamine/camphorsulfonic acid (43 μL of a THF solution that was 2 M in triethylamine and 1 M in camphorsulfonic acid) and tetrahydrofuran (0.29 mL) was added diethyl azodicarboxylate (11 μL, 0.072 mmol). This solution was maintained at 0° C. for thirty minutes, then allowed to warm to room temperature over 4 hours. The reaction then was concentrated to give a yellow residue. This residue was purified by HPLC (80/20/0.1 methanol/water/triflouroacetic acid; 90/10/0.1 methanol/water/triflouroacetic acid; C18 Luna) to give the C14 ester substituted with a 15-(allyloxy carbonyl) pentadecyloxy group (7.6 mg, 0.0094 mmol) in ca 90% purity and 32% yield. Spectra for this product matched those reported for this compound (Coffey, D. S.; McDonald, A. I.; Overman, L. E.; Rabinowitz, M. H.; Renhowe, P. A. J. Am. Chem. Soc. 2000, 20, 4893-4903.).

### Example 4

Preparation of 3,6-Dioxaoctyl ester

Example 3 is repeated with 11.54 mg (0.086 mmol) 3,6-dioxaoctanol [di(ethyleneglycol)ethyl ether] in place of allyl 16-hydroxy hexadecanoate to give the C14 ester substituted with a 3,6-dioxaoctyl group.

### Example 5

The following Example describes a method for synthesis of novel Crambescidin 657 analogs.

a. Representative Procedure for Attaching a Side Chain (—R₁—R₂) by Alkylation of the Crude Core Acid with an Alkyl Iodide in Benzene.

Formation of a Crambescidin 657 Derivative (Compound 20b)

A solution of triethylammonium formate (1 M in tetrahydrofuran, 0.17 mL, 0.17 mmol) was added to a solution of the formate salt of ester 18 (19 mg, 0.034 mmol), palladium tetrakis(triphenylphosphine) (11 mg, 0.010 mmol) and tetrahydrofuran (3.4 mL). This solution was maintained at room temperature for 16 hours, concentrated and the residue was dried in vacuo (0.05 torr, 6 h) to give the crude core acid as a yellow oil. This material was used without further purification.

Cesium carbonate (110 mg, 0.34 mmol) was added to a solution of the crude core acid, allyl 8-iodooctanoate (74 mg, 0.24 mmol) and benzene (1.1 mL, 0.03 M). After 5 minutes, silver nitrate (81 mg, 0.48 mmol) was added and the reaction mixture was stirred at room temperature for 12 hours. The crude reaction then was filtered through Celite (˜300 mg), which was washed with methylene chloride (˜8 mL). The filtrate was concentrated and the residue was purified on silica gel (10/1/1000 formic acid/iso-propanol/chloroform; 1/9 methanol/chloroform) to give crude 7-(allyloxycarbonyl)heptyl ester (compound 28b) as a 1:0.5 mixture with triphenylphosphine oxide. This mixture was carried forward without further purification.

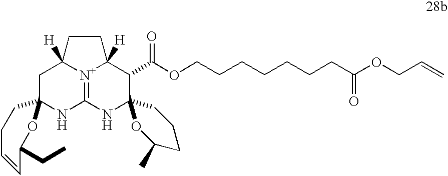

A solution of triethylammonium formate (1 M in tetrahydrofuran, 0.097 mL) was added to a solution of this sample of crude ester (compound 28b) obtained in the previous step, palladium tetrakis(triphenylphosphine) (11 mg, 0.0097 mmol) and tetrahydrofuran (0.97 mL). After 10 hours at room temperature, the reaction was concentrated onto Celite and the residue was purified on silica gel (10/5/1000 formic acid/iso-propanol/chloroform; 1/9 methanol/chloroform) to give 4.5 mg (0.0082 mmol, 24%) of C14 axially substituted 8-carboxyoctyl ester (compound 20b) as a yellow oil: ¹H NMR (500 MHz, CDCl₃) δ 5.65-5.69 (m, 1H), 5.48 (d, J=10.9 Hz, 1H), 4.49-4.53 (d, J=10.1 Hz, 1H), 4.29 (dt, J=9.9, 5.6 Hz, 1H), 4.17 (dt, J=10.2, 6.8 Hz, 1H), 3.98-4.03 (m, 2H), 3.86-3.93 (m, 1H), 2.93 (d, J=5.0 Hz, 1H), 2.62 (t, J=13.6 Hz, 1H), 2.56 (dd, J=12.7, 4.8 Hz, 1H), 2.10-2.38 (m, 9H), 1.87 (dd J=14.6, 5.2 Hz, 1H), 1.51-1.85 (m, 15H), 1.15-1.48 (m, 19H), 1.04 (d, J=6.2 Hz, 3H) 0.83 (t, J=7.2 Hz, 3H); ¹³C (125 MHz, CD₃OD) 177.9, 170.4, 150.4, 134.4, 131.5, 85.3, 82.3, 72.4, 68.6, 66.6, 55.7, 54.4, 50.9, 49.8, 49.7, 38.4, 38.0, 35.1, 33.2, 33.1, 32.7, 31.6, 31.0, 30.9, 30.8, 30.7, 30.6, 30.56, 30.4, 30.2, 30.0, 29.7, 27.7, 26.9, 26.3, 26.1, 24.6, 23.9, 21.9, 19.6, 14.6, 11.0 ppm; IR (film) 2930, 2856, 1733, 1656, 1610 cm⁻¹; HRMS: (ES) calcd for C₃₀H₄₈N₃O₆⁺ 546.3543, found: 546.3555

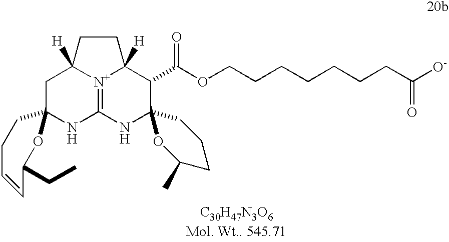

b. Representative Procedure for Attaching a Side Chain (—R₁—R₂) by Alkylation of the Crude Core Acid with an Alkyl Iodide in Methylene Chloride.

Preparation of Crambescidin 657 Derivative (Compound 20a)

A solution of triethylammonium formate (1 M in tetrahydrofuran, 0.20 mL, 0.20 mmol) was added to a solution of the formate salt of ester (compound 18) (22 mg, 0.039 mmol), palladium tetrakis(triphenylphosphine) (13 mg, 0.012 mmol) and tetrahydrofuran (3.9 mL). This solution was maintained at room temperature for 16 hours, concentrated and the residue was dried in vacuo (0.05 torr, 6 hours) to give the crude core acid as a yellow oil. This material was used without further purification.

Cesium carbonate (19 mg, 0.06 mmol) was added to a solution of this sample of the core acid, allyl 6-iodohexanoate (77 mg, 0.27 mmol) and methylene chloride (0.39 mL, 0.1 M). After 5 minutes, silver nitrate (20 mg, 0.12 mmol) was added and the reaction was stirred at room temperature for 16 hours and then filtered through Celite (˜300 mg), which was washed with methylene chloride (˜8 mL). The filtrate was concentrated and the residue was purified on silica gel (10/1/1000 formic acid/iso-propanol/chloroform; 1/9 methanol/chloroform) to give crude 5-(allyloxycarbonyl)pentyl ester (compound 28a) as a 1:0.5 mixture with triphenylphosphine oxide. This material was carried forward without further purification.

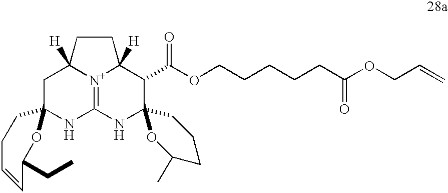

A solution of triethylammonium formate (1 M in tetrahydrofuran, 0.080 mL, 0.080 mmol) was added to a solution of this sample of ester obtained above, palladium tetrakis(triphenylphosphine) (9.0 mg, 0.008 mmol) and tetrahydrofuran (0.80 mL). This solution was maintained at room temperature for 10 hours. The reaction then was concentrated onto Celite and the residue was purified on silica gel (10/5/1000 formic acid/iso-propanol/chloroform; 1/9 methanol/chloroform) to give 11 mg (0.021 mmol, 54%) of crambescidin 657 derivative, 6-carboxyhexyl ester (compound 20a), as a yellow oil. ¹H NMR (500 MHz, CDCl₃) δ10.35 (br s, 1H), 10.02 (br s, 1H), 5.64-5.70 (m, 1H), 5.48 (d, J=10.2 Hz, 1H), 4.51 (d, J=8.7 Hz, 1H), 4.29 (dt, J=9.2, 5.13 Hz, 1H), 4.14-4.21 (m, 1H), 4.06-4.13 (m, 1H), 3.96-4.04 (m, 1H), 3.90-3.96 (m, 1H), 2.95 (d, J=5.0 Hz, 1H), 2.54-2.64 (m, 2H), 2.14-2.39 (m, 7H), 1.50-1.92 (m, 13H), 1.30-1.47 (m, 5H), 1.14-1.27 (m, 2H), 1.04 (d, J=6.2 Hz, 3H), 0.83 (t, J=7.2 Hz, 3H); ¹³C (125 MHz, CDCl₃) 177.0, 168.2, 148.8, 133.6, 129.9, 83.7, 80.7, 70.9, 67.1, 65.0, 53.9, 53.4, 51.9, 50.5, 37.0, 36.3, 35.2, 32.0, 31.8, 30.6, 29.7, 29.1, 28.2, 26.7, 25.2, 24.8, 23.6, 22.7, 21.5, 18.2, 14.1, 10.1 ppm; IR (film) 2926, 2856, 2362, 2339, 1733, 1656, 1613 cm⁻¹; HRMS: (ES) calcd for C₂₈H₄₄N₃O₆⁺ 518.3230, found: 518.3224.

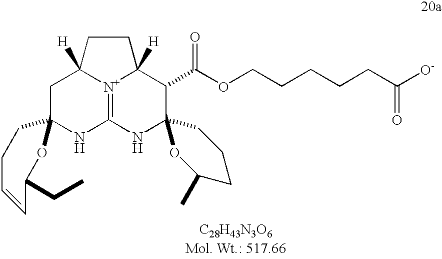

The yield and data for various novel Crambescidin 657 analogs are provided in Table 1.

TABLE 1
Yields and Data for Crambescidin 657 analogues

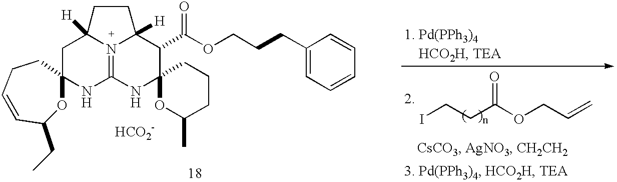

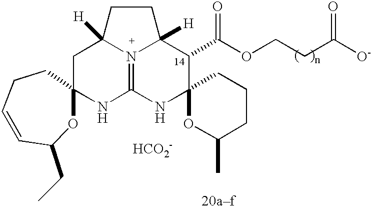

SN2 Products

| (n) | % Yield | HRMS | ¹HNMR-H₁₄ |
|---|---|---|---|
| 4 | 54 | 518.3224 | 2.95; d, J = 5.0Hz |
| 6 | 52 | 546.3543 | 2.93; d, J = 5.0 Hz |
| 8 | 57 | 574.3841 | 2.94; d, J = 4.9 Hz |
| 10 | 52 | 602.4155 | 2.93; d, J = 3.9Hz |
| 13 | 58 | 644.4658 | 3.07; d, J = 5.0Hz |
| 16 | 43 | 686.5123 | 3.09; d, J = 5.0Hz |

### Example 6

The following Example describes method for synthesizing novel Crambescidin 800 analogs.

Representative Procedure for Elaborating Side Chain Functionality. Preparation of Crambescidin 800 Derivatives from Crambescidin 657 Compounds (Compound 23).

Benzotriazol-1-yloxytris(dimethylamino)phosphonium hexafluorophosphate (17 mg, 0.037 mmol) was

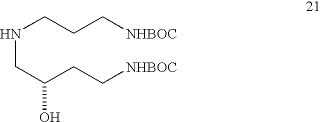

added to a solution of carboxypentyl ester (compound 20a), obtained under Example 5b, (13 mg, 0.025 mmol), di-N-(tert. Butoxycarbonyl) hydroxyspermidine (compound 21) (14 mg, 0.037 mmol), triethylamine (0.12 mL, 0.83 mmol) and methylene chloride (3.8 mL). After 7 hours, the reaction was diluted with ether (25 mL) and washed with saturated aqueous ammonium chloride (5 mL) and brine (5 mL). The organic layer was dried (magnesium sulfate), filtered and the residue was purified on silica gel (chloroform; 2/23 methanol/chloroform) to yield 25 mg of below mentioned carboxamide compound (compound 22a) contaminated with ˜0.8 equivalents of hexamethylphosphoramide. This sample was taken on without further purification.

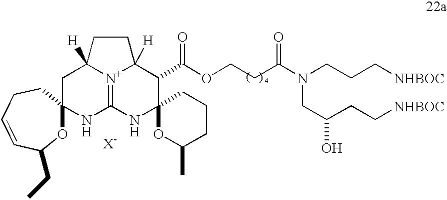

To a flask containing this sample of crude carboxamide compound (compound 22a) was added a solution of hydrochloric acid in ethyl acetate (2.9 M, 2.5 mL). This solution was maintained at room temperature for 30 minutes, and then concentrated in vacuo to give a yellow oil. The residue was purified by C18 reverse phase silica gel HPLC (95/5 methanol/0.1N sodium chloride in water) to give the contaminated with sodium chloride. This material was stirred with 50 mL of a 3:1 chloroform:methanol solution, filtered through a 4.5 μ filter and the residue was purified by silica gel chromatography (10/2/88 methanol/fornic acid/chloroform; 30/2/68 methanol/formic acid/chloroform) to give 11.0 mg (0.014 mmol, 58%) of Crambescidin 800 derivative (compound 23a; n=4) as a pale oil: ¹H NMR (500 MHz, CD₃OD, a mixture of amide rotamers) δ5.69-5.74 (m, 1H), 5.51 (d, J=11.0 Hz, 1H), 4.42 (d, J=9.5 Hz, 1H), 4.35 (dt, J=9.3, 5.5 Hz, 1H), 4.10-4.21 (m, 2H), 3.94-4.07 (m, 2H), 3.83-3.92 (m, 1H), 3.63-3.70 (m, 1H), 3.39-3.63 (m, 3 H), 3.33-3.35 (m, 0.5H), 3.20-3.28 (m, 0.7 H), 3.06-3.19 (m, 3.4H), 3.00 (t, J=7.6 Hz, 0.7 H), 2.85-2.96 (m, 1.6 H), 2.55-2.68 (m, 1.7H), 2.27-2.53 (m, 6H), 2.13-2.19 (m, 1.2 H), 2.05-2.12 (m, 0.4 H), 1.79-2.04 (m, 8H), 1.51-1.79 (m, 13H), 1.39-1.51 (m, 4.6H), 1.20-1.35 (m, 2H), 1.07 (d, J=6.2 Hz, 3H), 0.86 (t, J=7.3 Hz, 3 H); ¹³C (125 MHz, CDCl₃, a mixture of amide rotamers) 177.2, 176.2, 170.1, 150.3, 134.5, 131.5, 85.2, 82.2, 72.5, 69.6, 68.7, 68.6, 66.5, 55.7, 54.9, 54.0, 53.4, 51.4, 51.2, 48.0, 44.1, 38.7, 38.5, 38.4, 38.1, 34.1, 34.0, 33.2, 33.1, 32.9, 31.7, 30.4, 29.7, 28.0, 27.8, 27.0, 26.8, 26.2, 26.1, 24.6, 22.04, 21.95, 19.7, 11.0 ppm; IR (film) 3401, 3242, 2934, 1729, 1660, 1610 cm⁻¹; HRMS: (ES) calcd for C₃₅H₆₁N₆O₆⁺ 661.4653, found 661.4650.

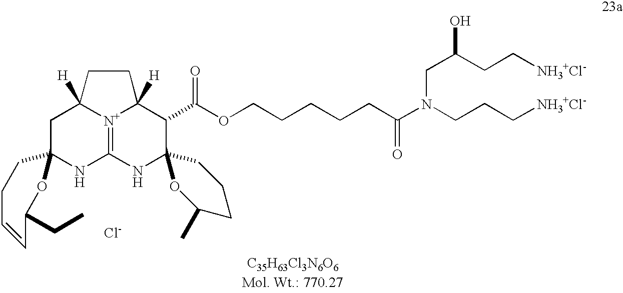

The yields and data for various novel crambescidin 800 analogs are provided in Table 2.

TABLE 2

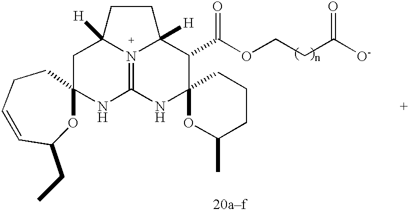

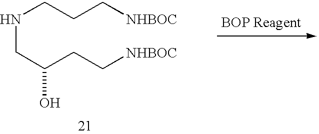

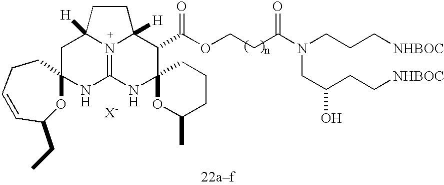

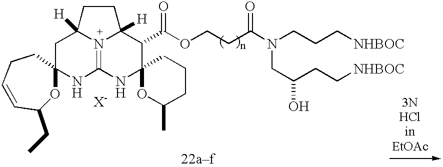

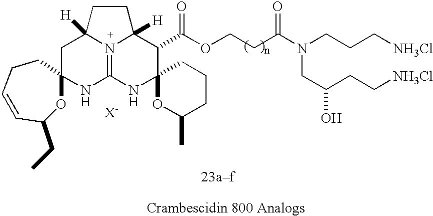

Yields and Data for Crambescidin 800 analogues

| | % Yield of | % Yield of | |
|---|---|---|---|
| (n) | Carboxamide | Crambescidin 800 | HRMS |
| 4 | 64 | 58 | 661.4650 |
| 6 | 49 | 26 | 689.4960 |
| 8 | 62 | quant | to be added |
| 10 | 57 | 22 | 745.5578 |
| 13 | 48 | 62 | 787.6052 |
| 16 | 59 | 23 | 829.6535 |

### Example 7

The following Example provides cytotoxicity data for novel Crambescidin 657 and 800 analogs.

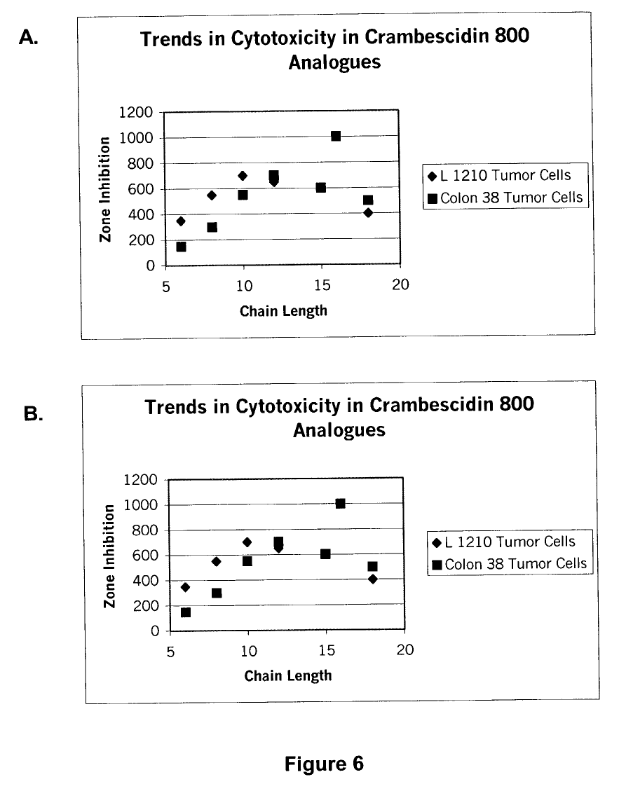

Disk diffusion based assay as described by Valeriote et al. (Int. J. Pharmacognosy (1995) 33, 59-66) was used for evaluating cytotoxicity of Crambescidin 657 and 800 analogs in an in vitro assay. Briefly, cells were embedded in an agar matrix, either as a monodisposed preparation directly from a murine tumor (e.g., colon 38 tumor, or as a single cell from a murine leukemia (L1210) cell line. The Crambescidin 657 (compounds 20a-f) and 800 (compounds 23a-f) analogs were placed on a filter disk, allowed to dry, and then placed on the agar and incubated. A zone of inhibition of colony growth was quantified as previously described. FIG. 6 illustrates cytotoxicity profiles of Crambescidin 657 and Crambescidin 800 analogs.

What is claimed is:

1. A pentacyclic compound of formula I: 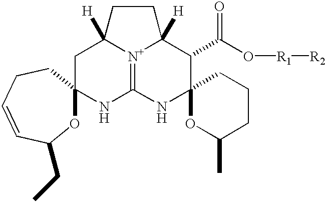
Wherein R1 is a hydrocarbyl group with from 1 to 20 atoms or is absent;
wherein R2 is absent or is selected from the group consisting of H, alkyl, aryl, heteroaryl, carboxy, carboxylate anion, phosphonate, phosphate, sulphonate, sulphate, borate, boronate and amine.

2. The pentacyclic compound of claim 1, wherein the hydrocarbyl group of R1 is selected from the group consisting of saturated, unsaturated, cyclic, acyclic, straight, branched chiral and achiral hydrocarbyl groups.

3. The pentacyclic compound of claim 1, wherein one or more carbon in R1 is replaced with one or more elements selected from O, S, or NR3, wherein R3 is alkyl, cycloalkyl or acyl.

4. The pentacyclic compound of claim 1, wherein one or more carbons in R1 is substituted with alkyl, aryl, aralkyl, heteroalkyl or a heteroaralkyl group.

5. The pentacyclic compound of claim 4, which further comprises one or more groups selected from the group consisting of halo, nitro, cyano, trifluoromethyl, hydroxy, thio, methylthio, amino, substituted amino, acylamino, aminoalkylamino, guanidino, carboxyl and carboalkoxy.

6. The pentacyclic compound of claim 1, wherein any one carbon in R1 is substituted with an aryl or heteroaryl group having from 6 to 14 carbon atoms.

7. The pentacyclic compound of claim 6, wherein the aryl or heteroaryl group is selected from the group consisting of phenyl, naphthyl, biphenylyl, furyl, thienyl, pyrrolyl, imidazolyl, benzofuranyl, benzoxazolyl, benzothiazolyl, thianaphthyl and indolyl.

8. The pentacyclic compound of claim 1, wherein the alkyl, aryl, heteroaryl group is substituted with one or more groups selected from the group consisting of halo, nitro, cyano, trifluoromethyl, hydroxy, thio, methylthio, amino, substituted amino, acylamino and guanidino.

9. The pentacyclic compound of claim 1, wherein R2 is an amine or amide.

10. The pentacyclic compound of claim 9, wherein the amine is NR₄R₅ or caroxamide-C0-NR₄R₅, where R₄ and R₅ are selected from the group consisting of H, alkyl, aralkyl, carboxyalkyl, amino-iminomethyl, hydroxyalkyl, acyl, aminoalkyl.

11. The pentacyclic compound of claim 10, wherein the amino-iminomethyl group is —C═N—R6(—NR7).

12. The pentacyclic compound of claim 11, wherein R6 and R7 is H or alkyl.

13. The pentacyclic compound of claim 12, wherein R6 and R7 form a cyclic structure.

14. The pentacyclic compound of claim 10, wherein the carboxyalkyl is substituted at the alkyl carbon by hydroxyalkyl, alkyl, thioalkyl, aminoalkyl, carboxyalkyl, amidoalkyl, guanidinoalkyl, aminoalkylaminoalkyl, hydroxyalkylaminoalkyl and acyl derivatives thereof.

15. The pentacyclic compound of claim 14, wherein the aminoalkyl is substituted with one or more hydroxy groups.

16. The pentacyclic compound of claim 10, wherein R₄ and R₅ combine to form a cyclic ring.

17. The pentacyclic compound of claim 16, wherein the cyclic ring is substituted with a heteroatom.

18. The pentacyclic compound of claim 16, wherein the heteroatom is O(morpholino).

19. The pentacyclic compound of claim 17, wherein the heteroatom is NR₈ where R₈ is selected from the group consisting of H(piperazino), alklyl (alkyl piperazino), carboxyalkyl, hydroxyalkyl and aminoalkyl.

20. The pentacyclic compound of claim 10, wherein the amino-iminomethyl has the formula —C═N—R₆(—NR₇), and R₆ and R₇ are selected from H and alkyl, or are bridged with 2 to 4 carbons to form a cyclic structure, provided that when R₂ is carboxylate anion or carboxamine, where —NR₄R₅ is hydroxyspermidine, then R₁ is not a saturated carbon chain with 16 carbons.

21. The compound of claim 1, wherein R₁ is —CH₂—CH═CH—, R₂ is phenyl.

22. The compound of claim 1, wherein R1 is H and R2 is absent.

23. The compound of claim 1, wherein R1 is alkyl, R2 is absent.

24. The compound of claim 23, wherein the alkyl group is decyl.

25. The compound of claim 1, wherein R1 is —(CH2-CH2-O—)n, R2 is alkyl.

26. The compound of claim 25, wherein n is 1-6, and R2 is ethyl.

27. The compound of claim 25, wherein R1 is —(CH2-CH2-O—)n, and R2 further comprises NR4R5.

28. The compound of claim 27, wherein n is 1-6 and R4 and R5 are H.

29. The compound of claim 27, wherein n is 1-6, and R4 and R5 are amino-iminomethyl of formula —C═N—R6(—NR7).

30. The compound of claim 29, wherein R6 and R7 are H or alky.

31. The compound of claim 30, wherein R6 and R7 form a cyclic structure.

32. The compound of claim 1, wherein R1 is a carboxyalkyl [CH2-(CH2)n] group, n=1-20; and R2 is an alkyl group.

33. The compound of claim 1, wherein where R1 is a carboxyalkyl [CH2-(CH2)n] group, n=1-20; and R2 is an allyl group.

34. A pentacyclic zwitterionic compound having the carboxylate side chain at C14 in natural axial orientation of the formula:

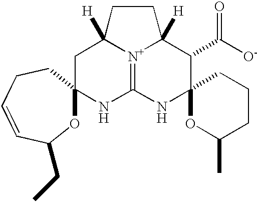

35. A pentacyclic compound of the formula: 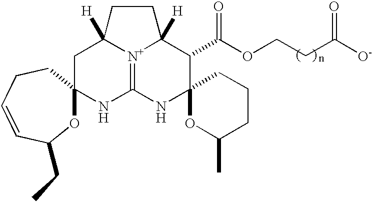
wherein, n=1-20

36. A pentacyclic compound of the formula: 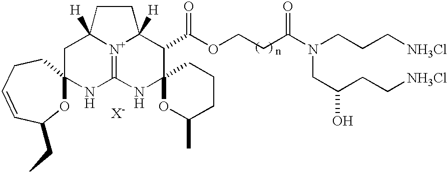
wherein,
n=1-20, and
X=any pharmaceutically acceptable counterion.

37. A pentacyclic compound of the formula: 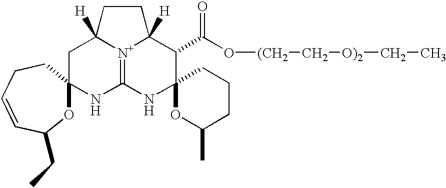
wherein, n=1-20

38. A pentacyclic compound of the formula: 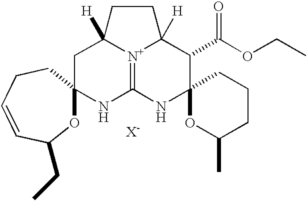
wherein, X=any pharmaceutically acceptable counterion.

39. A method for synthesizing the pentacyclic compound of claim 1 which comprises reacting a compound of formula: 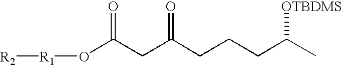
wherein, TBDMS is an alcohol protecting group with a compound of formula: 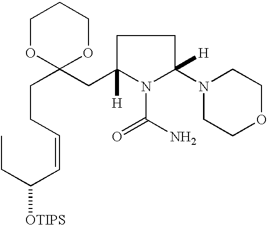
wherein TIPS is an alcohol protecting group to produce a compound of the formula: 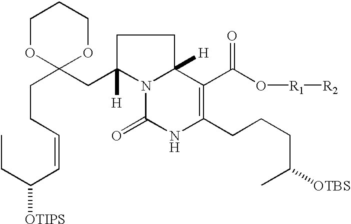
wherein TBPS and TIPS are alcohol protecting groups which is converted by deprotection, incorporation of ammonia, and cyclization to pentacyclic compound having the ester side chain in natural axial orientation of the formula: 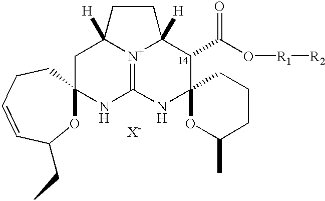
wherein X=any pharmaceutically acceptable counterion.

40. A method for synthesizing the pentacyclic compound of claim 21, wherein R1 is propenyl and R2 is phenyl
which comprises reacting a compound of formula: 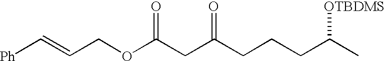
wherein, TBDMS is an alcohol protecting group with a compound of formula: 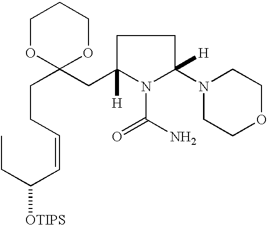
wherein TIPS is an alcohol protecting group to produce a compound of the formula: 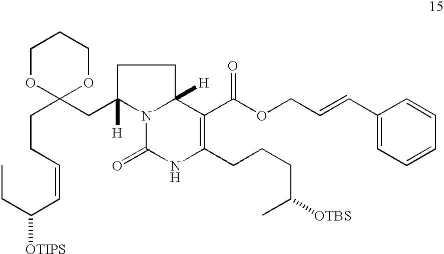
wherein TBPS and TIPS are alcohol protecting groups which is converted by deprotection, incorporation of ammonia, and cyclization to pentacyclic compound of claim 18 having the ester side chain in natural axial orientation of the formula: 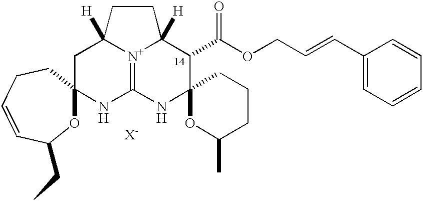
wherein, X=any pharmaceutically acceptable counterion.

41. A method of synthesizing a pentacyclic zwitterionic compound of the formula: 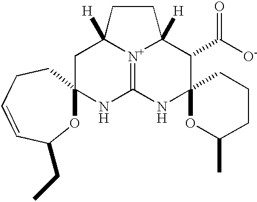
which comprises Palladium mediated deprotection of ester side chain of compound of claim 39 or 40.

42. A method of synthesizing crambescidin 431 of the formula: 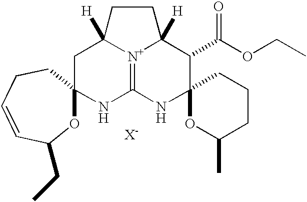
Wherein X=any pharmaceutically acceptable carrier, and
which comprises esterification of pentacyclic zwitterionic compound of claim 34 with ethanol.

43. A method for synthesizing an allyl ester side chain analog of the formula: 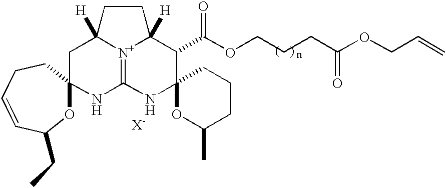
Wherein, n=1-20, and
X=any pharmaceutically acceptable counterion.
which comprises reacting the pentacyclic zwitterionic compound of claim 34 with an ω-iodoester of formula: 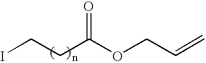
wherein n=1-20

44. A method of synthesizing a pentacyclic compound of the formula: 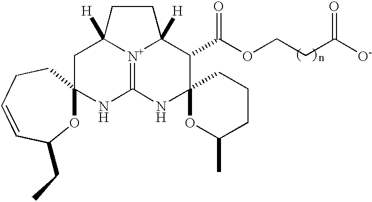
wherein n=1-20
which comprises palladium mediated deprotection of ester side chain of compound prepared by the method of claim 43.

45. A method of synthesizing a pentacyclic compound of the formula: 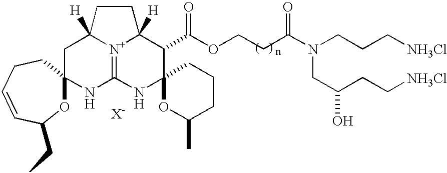
wherein, n=1-20, and
X=any pharmaceutically acceptable counterion.
which comprises reacting the reacting the pentacyclic compound of claim 34 with the compound of formula: 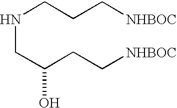
wherein BOC=an amine protecting group to produce a compound of the formula: 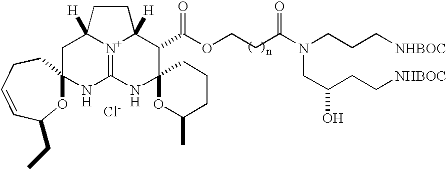
which is subsequently deprotected to produce a crambescidin 800 analog.

46. A method to synthesize the pentacyclic compound of claim 12 or 13, wherein the R1-R2 comprises a guanidino alkyloxy group of structure —(CH₂)n-NH═C—NH₂, comprising:
(a) treating the pentacyclic zwitterionic core acid of claim 34 with an amino alcohol of structure HO(CH2)n-NH2 to provide an amino alkyl ester; which is
(b) treated with alkyl-S—C—NH2(═NH) to produce the pentacyclic compound of claim 12 or 13, wherein R1-R2 comprises a guanidino alkyloxy group of structure —(CH₂)n-NH═C—NH₂

47. An antitumor composition comprising a compound of any one of claim 1 or 34-38 in admixture with a pharmaceutically acceptable carrier.

48. An antiviral composition comprising a compound of any one of claim 1 or 34-38 in admixture with a pharmaceutically acceptable carrier.

49. An antifungal composition comprising a compound of any one of claim 1 or 34-38 in admixture with a pharmaceutically acceptable carrier.

50. A Ca2+ channel blocker composition comprising a compound of any one of claim 1 or 34-38 in admixture with a pharmaceutically acceptable carrier.

51. A method for treating tumors comprising administering to a subject in need of said treatment, an amount of compound of any one of claim 1 or 34-38 effective to reduce the tumor load in the subject.

52. A method for treating viral infections comprising administering to a subject in need of said treatment, an amount of compound of any one of claim 1 or 34-38 effective to reduce the viral load in the subject.

53. A method for treating fungal infections comprising administering to a subject in need of said treatment, an amount of compound of any one of claim 1 or 34-38 effective to reduce the fungus load in the subject.
